(19) United States
(12) Patent Application Publication
(10) Pub. No.: US 2024/0335334 A1
(43) Pub. Date: Oct. 10, 2024

(54) ABSORBENT ARTICLES COMPRISING A LAYERED FLUID ACQUISITION/DISTRIBUTION SYSTEM AND METHODS FOR MAKING SAME

(71) Applicant: The Procter & Gamble Company, Cincinnati, OH (US)

(72) Inventors: Christopher Philip BEWICK-SONNTAG, Cincinnati, OH (US); John Daniel ALGERS, Montgomery, OH (US); Andrew R. LUMPKIN, Loveland, OH (US)

(21) Appl. No.: 18/628,984

(22) Filed: Apr. 8, 2024

Related U.S. Application Data
(60) Provisional application No. 63/458,197, filed on Apr. 10, 2023.

(51) Int. Cl. A61F 13/15; A61F 13/511

(52) U.S. Cl. CPC A61F 13/15577; A61F 13/51121; A61F 2013/15991

(57) ABSTRACT

Absorbent articles, for example baby diapers and/or training pants, feminine hygiene products, such as pads, and adult incontinence pads and/or pants, and more particularly absorbent articles that include an improved fluid acquisition/distribution system and methods for making same are provided.

### FIELD OF THE INVENTION

The present invention relates to absorbent articles, for example baby diapers and/or training pants, feminine hygiene products, such as pads, and adult incontinence pads and/or pants, and more particularly absorbent articles that comprise a layered fluid acquisition/distribution system and methods for making same.

### BACKGROUND OF THE INVENTION

Absorbent articles, e.g., diapers, training pants, feminine pads, adult incontinence pads, etc, are widely used among consumers. Generally, absorbent articles such as these comprise a topsheet and a backsheet with an absorbent core disposed therebetween. Some may include additional materials between the topsheet and the absorbent core or between the backsheet and the absorbent core to provide additional fluid management properties.

For such absorbent articles, it has proven advantageous to include various fluid handling systems such as a fluid acquisition/distribution system, for example a secondary topsheet, and/or a fluid storage system, such as an absorbent core. Such absorbent articles typically comprise one or more and typically two or more fluid handling systems, for example a fluid acquisition/distribution system and a fluid storage system. In addition to the fluid acquisition/distribution system and the fluid storage system, when present, other fluid handling systems may be included in the absorbent articles, such as a topsheet that typically is a soft, pliable, conformable material, for example fibrous structure, that contacts a user's skin and allows fluid, such as menstrual fluid, to easily pass through it in the z-direction readily permitting the fluid (once through the topsheet) to contact the fluid acquisition/distribution system (secondary topsheet) and oftentimes ultimately stored in the fluid storage system (for example absorbent core). The topsheet also maintains dryness of the user's skin. Further, in addition to the topsheet, secondary topsheet and absorbent core, other additional materials may be included in the absorbent articles, for example a tissue material, a material to provide resiliency to the products (bunching resistance), or a material to provide a better visual impression etc. as known in the art.

The various fluid handling systems may comprise various materials alone or in combination, such as filaments and/or fibers spun from polymeric materials, cellulose (e.g., wood pulp) fibers, and absorbent gelling materials (also known as “superabsorbent polymers”) (in fiber or particle form) (hereinafter, “AGM”). Various combinations and configurations of the various materials have been designed and manufactured for purposes of balancing competing objectives of effective fluid acquisition (capture) and retention, absorption capacity, user/wearer comfort (for example dryness and/or softness), minimization of bulkiness (volume, caliper, for example, for purposes of volumetric efficiency of packaging, user/wearer comfort and discreetness under clothing), material cost, and manufacturing case and efficiency.

Each of these types of fluid handling systems' materials has various features that provide both advantages and disadvantages. For example, accumulations of polymer filaments spun from polymers that are typically utilized in the various materials tend to be resilient and provide mechanical support and shape integrity to the absorbent article but are not particularly absorbent. Accumulations of cellulose fibers, for example pulp fibers, such as wood pulp fibers, generally have superior wicking (fluid distribution/transport) characteristics when densified and greater absorption capacity than accumulations of polymer filaments, but are substantially less resilient, particularly when wet, and tend to cause the absorbent article to lose shape integrity when wetted and to not feel soft cushiness to the touch of a user. Cellulose fibers, for example pulp fibers, such as wood pulp fibers, also tend to retain fluid on their surfaces, which if exposed to the skin of a wearer of a product including the fibers, can impart an unpleasant wet feeling to the absorbent article.

In general, absorbent articles are expected to absorb fluid, for example liquid, such as urine and/or menses, insults transferring the liquid from the point of insult on a topsheet of an absorbent article to an absorbent core of the absorbent article. Once the liquid insult is absorbed into the absorbent core, the absorbent article is expected to limit the amount of liquid which escapes the absorbent core and rewets the topsheet. For the acquisition of liquid insults, within a reasonable amount of time, the absorbent core or an additional material, such as a fluid acquisition/distribution system, oftentimes referred to as a secondary topsheet, between the topsheet and the absorbent core should be in liquid contact with the topsheet, for example where a pre-formed topsheet is in physical contact with a pre-formed secondary topsheet, to adequately drain the topsheet of the liquid insult and ultimately transverse through the fluid acquisition/distribution system (secondary topsheet) ultimately being stored in the absorbent core (fluid storage system).

However, variables affecting acquisition speed can be diametrically opposed to rewet performance. For example, some conventionally known topsheets require a trade-off between capillarity, permeability, and rewet properties. So, while good liquid acquisition can be achieved by making the topsheet hydrophilic so that a fluid passes through the topsheet quickly, the topsheet then typically suffers from poor rewet performance. The converse is also true as a hydrophobic topsheet may provide better rewet performance; however, fluid acquisition times will likely increase due to the hydrophobic nature of the topsheet.

In addition to fluid acquisition and rewet performance properties of an absorbent article, an absorbent article is expected to provide the user with a comfortable feel. Particularly in the context of feminine hygiene articles or feminine adult incontinence articles, this can be a real challenge. While some absorbent articles may be created to provide great conformity to the intricate female anatomy, such conformity can reduce the structural integrity of the absorbent article. The reduced structural integrity of the absorbent article can cause bunching during use and also inhibit recovery of the absorbent article to its original form. Unfortunately, the bunching of the absorbent article can lead to discomfort for the wearer and/or leakage during use.

As such, it would be beneficial to have an improved absorbent article which addresses the tradeoff of comfortable conformance and resiliency as well as one that provides good fluid kinetics, for example fluid acquisition without negative rewet performance. It would also be beneficial to provide a method for creating such absorbent articles and/or components thereof without sacrificing leakage performance of the absorbent article.

In absorbent articles, the fluid acquisition/distribution system (secondary topsheet) is typically placed on top of the body facing surface of a fluid storage system (absorbent core). The fluid acquisition/distribution system has the function of rapidly acquiring fluids excreted and/or discharged from the body and transferring them rapidly away from the body's skin, in one example the fluid acquisition/distribution system draws fluid from the fluid source through a topsheet, when present, that is in contact with the body's skin into the fluid acquisition/distribution system, which may, in one example, then release the fluid into a fluid storage system.

The fluid acquisition/distribution system also functions to keep the fluid storage system, when present, distanced from the body's skin to avoid excreted and/or discharged body fluids rewetting the body's skin while a user is wearing/using the absorbent article. In some cases, the fluid acquisition/distribution system, in addition to acquiring and transferring body fluids away from the body's skin (for example z-direction transfer of fluids) also functions to distribute the body's fluids on a larger surface area (for example x-y direction distribution of fluids) so to provide a more efficient usage of the surface of the fluid storage system, when present.

It is believed that users of absorbent articles, such as feminine hygiene pads in particular, prefer wearer-facing surface staining from fluid discharge (for example menstrual flow) to be as small (in the x-y direction) as possible, which signals to the user that the absorbent article has rapidly drawn discharged fluid into the fluid acquisition/distribution system and ultimately absorbed the fluid into an absorbent core, and has effectively contained it, rather than allowing fluid to spread and possibly escape the absorbent article.

Attempting to take advantage of beneficial features of these typical fluid handling systems' materials while mitigating their disadvantages, manufacturers of absorbent articles have developed various configurations in which the absorbent article's fluid handling systems are arranged. However, current known configurations have left room for improvement in fluid handling, such as fluid absorption and importantly fluid partitioning to a fluid storage system, for example an absorbent core, controlling fluid movement along x- and y-directions versus z-direction along or proximate wearer-facing surfaces while simultaneously delivering a high tactile cushiness, softness, flexibility, wet collapse resistance and resiliency.

Fluid handling systems' materials are configured and/or arranged to achieve a capillarity cascade where the capillarity increases moving away from a body's skin with an initial acquisition in an acquisition material then at least partially moving to a distribution material, and then at least partially to a storage material, when present. Capillarity of a material is a function of the density of the material and the components making up the material.

Known fluid acquisition/distribution systems have utilized pre-formed, discrete materials that are superimposed on each other to form the fluid acquisition/distribution system. A primary function of fluid acquisition/distribution systems is to acquire fluid, for example menstrual fluid, and are considered temporary volume in the absorbent article that can capture an initial insult of fluid. After capture of a fluid by a fluid acquisition/distribution system, the captured fluid needs to be distributed to a fluid storage system, if present, and/or a fluid storage portion of the fluid acquisition/distribution system away from the initial fluid loading site of the fluid acquisition/distribution system to ensure that the initial fluid loading site is then available for a subsequent round of fluid loading, for example fluid insult.

Oftentimes these pre-formed, discrete materials are in the form of separate pre-formed fibrous structures. One problem with an arrangement of these separate pre-formed fibrous structures is that each pre-formed fibrous structure invariably competes with the other pre-formed fibrous structures so the properties of each pre-formed fibrous structure for moving a fluid from a first pre-formed fibrous structure, for example a topsheet, when present, to a second pre-formed fibrous structure, for example a fluid acquisition/distribution system (secondary topsheet), and then to a third pre-formed fibrous structure, for example a fluid storage system (absorbent core), when present, has unintended side effects. For example, to move a fluid the capillarity force necessary to move the fluid from a topsheet to a fluid acquisition/distribution system (secondary topsheet), the fluid acquisition/distribution system's capillarity needs to be substantially higher than the topsheet's capillarity. A similar relationship exists between a fluid acquisition/distribution system, for example a secondary topsheet, and a fluid storage system, for example an absorbent core, where the capillarity of the fluid storage system needs to be substantially higher than the capillarity of the fluid acquisition/distribution system. A fibrous structure's capillarity is driven by high surface area of fibers/filaments to void volume (i.e., higher density gradient as fluid moves from topsheet to fluid acquisition/distribution system to fluid storage system). This densification gradient (referred to as “capillarity cascade”) means increasing densifying the materials and/or fibrous structures from the topsheet, when present, to the fluid acquisition/distribution system, for example a secondary topsheet, to a fluid storage system, when present. Densification of the materials and/or fibrous structures results in stiffening, loss of flexibility, loss of conformability (to the user's body shape) of the materials and/or fibrous structures such that the topsheet, when present, is less dense than the fluid acquisition/distribution system which is less dense than the fluid storage system, when present.

Further, another negative associated with forming absorbent articles with multiple pre-formed materials, for example pre-formed topsheets, pre-formed secondary topsheets, pre-formed absorbent cores, and/or pre-formed backsheets that are stacked together in an arrangement to form the absorbent articles is the cost and complexities associated with one or more additional transformations need for the multiple pre-formed materials, such as winding and unwinding, laminating and then winding up again after laminating.

One approach in the past to mitigate these limitations of moving fluid between pre-formed, discrete fibrous structures in a multi-fibrous structure fluid handling system has been to create a unitary, integrated fluid acquisition/distribution system comprising an airlaid fibrous structure, for example an airlaid nonwoven substrate having a cellulose rich layer. The airlaid fibrous structure is formed by depositing cellulose and potentially binder fibers, for example synthetic binder fibers, onto a pre-formed nonwoven substrate under vacuum air flow to better embed the cellulose fibers and optionally binder fibers within the pre-formed nonwoven substrate and reduce boundary effects as disclosed in U.S. Patent Application Publication No. 2018/0094227 A1. However, it is challenging to control the properties (density, capillarity) of cellulose rich layer that is deposited onto the pre-formed nonwoven substrate as both materials inherently compress and densify during manufacture and/or during storage and particularly when prepared for efficient bulk shipment such as in a roll or festooned format where material at the inner core of the roll or bottom of a festoon box experience more compression than in the external (outer) layers of the roll or the festoon box. This uncontrolled densification of the unitary, integrated fluid acquisition/distribution system can increase the capillarity of the unitary, integrated fluid acquisition/distribution system to a level that hinders its drainage into a fluid storage system adjacent thereto.

Formulators have addressed this problem of uncontrolled densification by including synthetic staple fibers (typically BiCo fibers or PET) that are seeded within the absorbent article or fibrous structures within the absorbent article, for example a fluid acquisition/distribution system, to help sustain a target caliper or enable recovery from uncontrolled compression but in order to be effective synthetic staple fibers need to be thick (larger average diameter), and ideally bonded together leveraging typically thermal heat energy to create a structurally stable network. Such thick synthetic staple fibers typically exhibit average diameters of at least 15 μm to about 40 μm and relatively stiff when bonded to form a resilient network to resist uncontrolled densification. Unfortunately, the inclusion of such synthetic staples fibers having average diameters of at least 15 μm results in a stiff (as measured according to the Wet and Dry CD and MD 3 Point Bend Test Method described herein) absorbent article and/or fibrous structure of the absorbent article, for example the fluid acquisition/distribution system, that negatively impacts a user's in-use comfort. The bending stiffness of a synthetic staple fiber is proportional to the cubed of its average diameter. For example, a synthetic staple fiber having an average diameter of 20 μm is 8 times stiffer to bend than the same fiber that exhibits an average diameter of 10 micron.

In addition to the negatives associated with the use of synthetic staple fibers having average diameters of at least 15 μm and densification of materials and/or fibrous structures of the absorbent articles, another negative result of combining pre-formed, discrete materials, for example separate fibrous structures that are laid on top of one another, bonded or unbonded, an air gap is formed between the adjacent pre-formed, discrete materials, for example separate pre-formed fibrous structures at their respective interfaces. This air gap acts as a boundary, for example an air-boundary-layer, that hinders capillarity driven fluid transport between the adjacent pre-formed, discrete materials, for example separate pre-formed fibrous structures.

Formulators have attempted to remove the air-boundary-layer by integrating the layers of materials to minimize the size of any air-boundary-layers within the fluid handling systems. As a result, formulators have made unitary fluid handling systems, for example unitary fluid acquisition/distribution systems and fully unitary fluid storage systems that have fluid acquisition, fluid distribution and fluid storage layers all integrated into a unitary structure, examples of which are described in U.S. Patent Application Publication No. 2018/0094227 A1. However, such unitary fluid handling systems exhibit negatives for consumers and manufacturers. Unitary structures are not easy to make or to control the final properties of. For example, a typical unitary core structure, examples of which are described in U.S. Patent Application Publication No. 2018/0094227 A1, was made on an airlaid process that improves the fluid handling of the absorbent system. Airlaid production lines typically have three or more forming or fiber/AGM laydown stations in series. The airlaid process utilizes short fiber systems (less than 2.5 mm cellulose or less than 6 mm for synthetic fibers typically) and generally need to be densified to maintain integrity during storage and reduce transport costs, which results in the unitary core structure exhibiting unacceptable stiffness (bending stiffness) that lacks conformability to her body shape.

Regarding comfort, some consumers may desire a product that has sufficient thickness and stiffness to provide the desirable amount of protection while also being flexible. Lofty materials may be utilized to provide a thick cushiony feeling absorbent article. However, lofty materials can experience a variety of compressive loads that result in undesirable compression, without recovery in the raw (pre-production) material handling, product making and finally in-use due to bodily forces. Recovery from these compressive loads is paramount in maintaining the cushiony feeling of the article. Exacerbating this issue is the fact that the characteristics of the materials of the absorbent article change once fluid is introduced into the absorbent article. Hence, an absorbent article that may meet a consumer's requisite criteria before use may no longer be comfortable, flexible, conformable or have the desired stiffness to the user after a given amount of fluid has been absorbed by the absorbent article.

Therefore, there is a need to be able to create a unitary fluid acquisition/distribution system that is able to resist or recover from manufacturing, storage and transport compression packaging while preserving its targeted densification while maintaining its soft, conformable mechanical properties that allow it to be both soft to the touch and gently mold to her individual genital anatomical shape to better capture fluid as it exits her body.

As such there is a need to create fluid management materials, for example a fluid acquisition/distribution system, that have sufficient caliper and compressive recovery for use in absorbent articles.

An ideal fluid acquisition/distribution system typically is formed from a material that performs the function of a high masking, fast acquiring yet efficient draining (for example z-direction transfer of fluids) while still exhibiting a high wet compressive resiliency and/or recovery and that also performs the function of distributing the excreted and/or discharged body fluids over a larger surface area (for example x-y direction distribution of fluids) so to provide a more efficient usage of the surface of the fluid storage system (fluid storage layer).

There is a desire to make a lofty, cushiony, conformable fluid acquisition/distribution system that functions to acquire and distribute fluids effectively and oftentimes partition and drain fluids into a fluid storage system, when present in an absorbent article.

Accordingly, there is a need for fluid acquisition/distribution systems that overcome the negatives associated with known fluid acquisition/distribution systems, absorbent articles comprising same and methods for making same.

### SUMMARY OF THE INVENTION

The present invention fulfills the need described above by providing novel layered fluid acquisition/distribution systems that overcome the negatives of known fluid acquisition/distribution systems, absorbent articles comprising the novel layered fluid acquisition/distribution systems and methods for making such novel layered fluid acquisition/distribution systems.

One solution to solve the problems associated with known absorbent articles formed from multiple pre-formed materials, for example pre-formed topsheets, pre-formed secondary topsheets, pre-formed absorbent cores, and/or pre-formed backsheets that are stacked together in an arrangement to form the absorbent articles is to create novel layered fluid acquisition/distribution systems, which may function and/or be considered as novel secondary topsheets, described herein. The novel layered fluid acquisition/distribution systems reduce the number of pre-formed materials being used to make absorbent materials, for example by forming the novel layered fluid acquisition/distribution systems comprising a first layer comprising a base nonwoven substrate and a second layer comprising a coform fibrous structure formed directly on the base nonwoven substrate, for example by spinning a plurality of filaments forming at least a part of the coform fibrous structure such that the plurality of filaments, with or without fibers mixed therewith, are directly laid on a surface of the base nonwoven substrate. Such novel layered fluid acquisition/distribution systems at a minimum reduce the complexities and costs associated with known absorbent articles comprising multiple pre-formed materials described above in addition to providing improved performance benefits and/or consumer benefits.

In one example of the present invention, a fluid acquisition/distribution system, for example a layered fluid acquisition/distribution system, for example a unitary, layered fluid acquisition/distribution system, comprising:
a. a first layer comprising a base nonwoven substrate, for example a carded base nonwoven substrate and/or a spunlace base nonwoven substrate and/or a hydroentangled base nonwoven substrate and/or a spunbond base nonwoven substrate; and
b. a second layer comprising a coform fibrous structure, wherein the coform fibrous structure comprises a plurality of filaments and a plurality of fibers, for example wherein the plurality of filaments and the plurality of fibers are commingled together, wherein the plurality of filaments are spun from a die, mixed with a plurality of fibers to form a mixture of filaments and fibers and the mixture is then directly laid and/or deposited on a surface of the base nonwoven substrate such that a coform fibrous structure formed from the mixture of filaments and fibers is formed on the surface of the base nonwoven substrate (alternatively wherein the coform fibrous structure is a pre-formed coform fibrous structure that is then associated with, for example thermally bonded to adhesively bonded to, and/or chemically bonded to, the base nonwoven substrate), is provided.

In one example of the present invention, a fluid acquisition/distribution system, for example a layered fluid acquisition/distribution system, for example a unitary, layered fluid acquisition/distribution system, comprising:
a. a first layer comprising a base nonwoven substrate, for example a carded base nonwoven substrate and/or a spunlace base nonwoven substrate and/or a hydroentangled base nonwoven substrate and/or a spunbond base nonwoven substrate; and
b. a second layer comprising a pre-formed coform fibrous structure, wherein the pre-formed coform fibrous structure comprises a plurality of filaments and a plurality of fibers, for example wherein the plurality of filaments and the plurality of fibers are commingled together, wherein the pre-formed coform fibrous structure is associated with, such as bonded to, for example thermally bonded and/or to adhesively bonded to and/or chemically bonded to, a surface of the base nonwoven substrate, is provided.

In another example of the present invention, a process for making a layered fluid acquisition/distribution system, for example a unitary, layered fluid acquisition/distribution system, the process comprising the steps of:
a. providing a base nonwoven substrate, for example a carded base nonwoven substrate and/or a spunlace base nonwoven substrate and/or a hydroentangled base nonwoven substrate and/or a spunbond base nonwoven substrate;
b. spinning a plurality of filaments from a die;
c. mixing a plurality of fibers with the plurality of filaments such that the filaments and fibers are commingled together forming a mixture of filaments and fibers, such as a coform mixture;
d. collecting, for example directly collecting, the coform mixture on a surface of the base nonwoven substrate such that a coform fibrous structure is formed on the surface of the base nonwoven substrate, producing a layered fluid acquisition/distribution system, for example a unitary, layered fluid acquisition distribution system, is provided.

In another example of the present invention, a process for making a layered fluid acquisition/distribution system, for example a unitary, layered fluid acquisition/distribution system, the process comprising the steps of:
a. providing a base nonwoven substrate, for example a carded base nonwoven substrate and/or a spunlace base nonwoven substrate and/or a hydroentangled base nonwoven substrate and/or a spunbond base nonwoven substrate;
b. providing a coform fibrous structure comprising a plurality of filaments commingled with a plurality of fibers;
c. associating the coform fibrous structure with a surface of the base nonwoven substrate by bonding, for example by thermally bonding and/or adhesively bonding and/or chemically bonding, the coform fibrous structure to a surface of the base nonwoven substrate such that a layered fluid acquisition/distribution system, for example a unitary, layered fluid acquisition/distribution system if formed, is provided.

In yet another example of the present invention, a core structure comprising a layered fluid acquisition/distribution system of the present invention and a fluid storage system, for example an absorbent core, and optionally a topsheet, wherein the layered fluid acquisition/distribution system is positioned between the topsheet, when present, and the fluid storage system, is provided.

In still another example of the present invention, an absorbent article, for example a baby diaper and/or training pant, feminine hygiene pads, and adult incontinence pads and/or pants, comprising a topsheet, a layered fluid acquisition/distribution system of the present invention, and a fluid storage system, for example an absorbent core, is provided.

In still yet another example of the present invention, an absorbent article, for example a baby diaper and/or training pant, feminine hygiene pads, and adult incontinence pads and/or pants, comprising a topsheet, a layered fluid acquisition/distribution system of the present invention, and a fluid storage system, for example an absorbent core, wherein the layered fluid acquisition/distribution system is positioned between the topsheet and the fluid storage system, is provided.

In even another example of the present invention, an absorbent article, for example a baby diaper and/or training pant, feminine hygiene pads, and adult incontinence pads and/or pants, comprising a layered fluid acquisition/distribution system of the present invention, and optionally, a fluid storage system, for example an absorbent core, wherein the absorbent article exhibits a MD Dry Bending Stiffness of less than 40 and/or less than 38 and/or less than 35 and/or less than 30 and/or less than 25 and/or less than 20 and/or less than 15 to about 10 N*mm², specifically including all values within these ranges and any ranges created thereby, as measured according to the Wet and Dry CD and MD 3 Point Bend Test Method described herein, is provided.

In even yet another example of the present invention, an absorbent article, for example a baby diaper and/or training pant, feminine hygiene pads, and adult incontinence pads and/or pants, comprising a topsheet, a layered fluid acquisition/distribution system of the present invention, and a fluid storage system, for example an absorbent core, wherein the layered fluid acquisition/distribution system is positioned between the topsheet and the fluid storage system, for example an absorbent core, wherein the absorbent article exhibits a MD Dry Bending Stiffness of less than 40 and/or less than 38 and/or less than 35 and/or less than 30 and/or less than 25 and/or less than 20 and/or less than 15 to about 10 N*mm², specifically including all values within these ranges and any ranges created thereby, as measured according to the Wet and Dry CD and MD 3 Point Bend Test Method described herein, is provided.

In even another example of the present invention, an absorbent article, for example a baby diaper and/or training pant, feminine hygiene pads, and adult incontinence pads and/or pants, comprising a layered fluid acquisition/distribution system of the present invention, and optionally, a fluid storage system, for example an absorbent core, wherein the absorbent article exhibits a 5th cycle wet energy of recovery less than 4 N*mm and/or less than 3 N*mm and/or less than about 2.5 N*mm and greater than about 1 N*mm, specifically including all values within these ranges and any ranges created thereby, as measured according to the Wet and Dry Bunched Compression Test Method described herein, is provided.

In even yet another example of the present invention, an absorbent article, for example a baby diaper and/or training pant, feminine hygiene pads, and adult incontinence pads and/or pants, comprising a topsheet, a layered fluid acquisition/distribution system of the present invention, and a fluid storage system, for example an absorbent core, wherein the layered fluid acquisition/distribution system is positioned between the topsheet and the fluid storage system, for example an absorbent core, wherein the absorbent article exhibits a 5th cycle wet energy of recovery less than 4 N*mm and/or less than 3 N*mm and/or less than about 2.5 N*mm and greater than about 1 N*mm, specifically including all values within these ranges and any ranges created thereby, as measured according to the Wet and Dry Bunched Compression Test Method described herein, is provided.

In even another example of the present invention, an absorbent article, for example a baby diaper and/or training pant, feminine hygiene pads, and adult incontinence pads and/or pants, comprising a layered fluid acquisition/distribution system of the present invention, and optionally, a fluid storage system, for example an absorbent core, wherein the absorbent article exhibits a 5th cycle wet % recovery greater than 30% and/or greater than 35% and/or greater than 40% and/or greater than 45% and/or greater than 50% and/or from about 35% to about 60%, specifically including all values within these ranges and any ranges created thereby, as measured according to the Wet and Dry Bunched Compression Test Method described herein, is provided.

In even yet another example of the present invention, an absorbent article, for example a baby diaper and/or training pant, feminine hygiene pads, and adult incontinence pads and/or pants, comprising a topsheet, a layered fluid acquisition/distribution system of the present invention, and a fluid storage system, for example an absorbent core, wherein the layered fluid acquisition/distribution system is positioned between the topsheet and the fluid storage system, for example an absorbent core, wherein the absorbent article exhibits a 5th cycle wet % recovery greater than 30% and/or greater than 35% and/or greater than 40% and/or greater than 45% and/or greater than 50% and/or from about 35% to about 60%, specifically including all values within these ranges and any ranges created thereby, as measured according to the Wet and Dry Bunched Compression Test Method described herein, is provided.

The present invention provides a novel layered fluid acquisition/distribution system, for example a unitary layered fluid acquisition/distribution system, an absorbent core structure comprising a novel layered fluid acquisition/distribution system and a fluid storage system, an absorbent article comprising a novel layered fluid acquisition/distribution system and a method for making a novel layered fluid acquisition/distribution system, an absorbent core structure comprising a novel layered fluid acquisition/distribution system and an absorbent article comprising a novel layered fluid acquisition/distribution system.

### BRIEF DESCRIPTION OF THE DRAWINGS

FIG. 1 is a schematic, plan view representation of an example of an absorbent article according to the present invention;

FIG. 2A is a schematic, lateral cross section of the absorbent article of FIG. 1, taken through line 2A-2A in FIG. 1.

FIG. 2B is a schematic representation of an enlarged view of a portion of FIG. 2A, taken within circle 2B in FIG. 2A.

FIG. 3 is a cross-sectional representation of an example of a layered fluid acquisition/distribution system according to the present invention;

FIG. 4 is a cross-sectional representation of another example of a layered fluid acquisition/distribution system according to the present invention;

FIG. 5 is a schematic representation of an example of a process of the present invention for making a layered fluid acquisition/distribution system of the present invention that utilizes two-sided fiber injection, but a single-sided fiber injection may alternatively be utilized;

FIG. 6 is a schematic representation of another example of a process of the present invention for making a layered fluid acquisition/distribution system of the present invention that utilizes two-sided fiber injection, but a single-sided fiber injection may alternatively be utilized;

FIG. 7 is a schematic representation of an example of a filament-forming hole and fluid-releasing hole from a suitable die useful in making a layered fluid acquisition/distribution system of the present invention;

FIG. 8A is a schematic representation of an example of a setup of equipment used in measuring the Wet and Dry Bunched Compression of absorbent articles according to the Wet and Dry Bunched Compression Test Method;

FIG. 8B is a schematic representation of a component of the equipment shown in FIG. 8A in a first state;

FIG. 8C is a schematic representation of the component of the equipment shown in FIG. 8B in a second state;

FIG. 9A a representative graph of force (N) versus displacement or extension (mm) created by data from all 5 cycles from the Wet and Dry Bunched Compression Test Method; and

FIG. 9B a representative graph of force (N) versus displacement or extension (mm) showing Dry Energy Compression for Cycle 1, Dry Energy Loss from Cycle 1 and the Dry Energy of Recovery for Cycle 1 created by data from Cycle 1 from the Wet and Dry Bunched Compression Test Method.

### DETAILED DESCRIPTION OF THE INVENTION

### Definitions

“Fibrous structure” as used herein means a structure that comprises a plurality of fibrous elements, for example a plurality of filaments and/or a plurality of fibers. In addition to the filaments and/or fibers, the fibrous structures may comprise other materials such as particles, for example SAP particles, and/or pulp fibers. In one example, a fibrous structure according to the present invention means an orderly arrangement of filaments and particles within a structure in order to perform a function, for example absorb liquids. In another example, a fibrous structure according to the present invention is a nonwoven. In one example, the fibrous structures of the present invention may comprise coform fibrous structures, meltblown fibrous structures, and spunbond fibrous structures so long as they contain particles. In one example, the fibrous structure is a non-hydroentangled fibrous structure. In another example, the fibrous structure is a non-carded fibrous structure.

In another example of the present invention, a fibrous structure comprises a plurality of fibrous elements, for example a plurality of inter-entangled fibrous elements, for example inter-entangled filaments, and optionally a plurality of fibers, dispersed between the inter-entangled filaments.

The fibrous structures of the present invention may be homogeneous, non-homogeneous, or layered. If layered, such as the layered fluid acquisition/distribution system, and/or fibrous structure components of the absorbent articles of the present invention, the fibrous structures may comprise at least two and/or at least three and/or at least four and/or at least five layers of materials, for example fibrous elements and/or particles.

The fibrous structures of the present invention may exhibit basis weights of from about 10 gsm to about 400 gsm and/or from about 10 gsm to about 350 gsm and/or from about 20 to about 300 gsm and/or from about 30 gsm to about 250 gsm and/or from about 40 gsm to about 200 gsm and/or from about 40 gsm to about 150 gsm and/or from about 40 gsm to about 125 gsm and/or from about 40 to about 100 gsm, specifically including all values within these ranges and any ranges created thereby, as measured according to the Basis Weight Test Method described herein. In one example, the fibrous elements, for example filaments and/or fibers, are present in the fibrous structures of the present invention at a basis weight of from about 10 gsm to about 400 gsm and/or from about 10 gsm to about 350 gsm and/or from about 20 to about 300 gsm and/or from about 30 gsm to about 250 gsm and/or from about 40 gsm to about 200 gsm and/or from about 40 gsm to about 150 gsm and/or from about 40 gsm to about 125 gsm and/or from about 40 to about 100 gsm, specifically including all values within these ranges and any ranges created thereby, as measured according to the Basis Weight Test Method described herein. In one example, the particles, when present, for example SAP particles, are present in the fibrous structures of the present invention at a basis weight of from about 10 gsm to about 200 gsm and/or from about 10 gsm to about 150 gsm and/or from about 20 to about 150 gsm and/or from about 30 gsm to about 100 gsm and/or from about 40 gsm to about 60 gsm, specifically including all values within these ranges and any ranges created thereby, as measured according to the Basis Weight Test Method described herein.

“Multi-fibrous element fibrous structure” as used herein means a fibrous structure that comprises filaments and fibers, for example a coform fibrous structure is a multi-fibrous element fibrous structure.

“Mono-fibrous element fibrous structure” as used herein means a fibrous structure that comprises only fibers or filaments, for example a meltblown fibrous structure, such as a scrim, respectively, not a mixture of fibers and filaments.

“Coform fibrous structure” as used herein means that the fibrous structure comprises a mixture of filaments, such as filaments, for example meltblown filaments, such as thermoplastic filaments, for example polypropylene filaments, and fibers, for example pulp fibers, such as wood pulp fibers, and/or staple fibers, and optionally SAP particles. In one example, the filaments and fibers, for example pulp fibers, and optionally, particles, for example SAP particles, are commingled together to form the coform fibrous structure. The coform fibrous structure may be associated with one or more meltblown fibrous structures and/or spunbond fibrous structures, which form a scrim (or scrim layer that is deposited, for example spun directly onto a surface of a fibrous structure of the present invention that is being concurrently formed or that is already pre-formed and/or spun directly onto a collection device prior to a fibrous structure of the present invention being formed (via spinning) directly on a surface of the scrim layer (in one example the scrim may be present at a basis weight of greater than 0.5 gsm to about 5 gsm and/or from about 1 gsm to about 4 gsm and/or from about 1 gsm to about 3 gsm and/or from about 1.5 gsm to about 2.5 gsm, specifically including all values within these ranges and any ranges created thereby, as measured according to the Basis Weight Test Method described herein), such as on one or more surfaces of the coform fibrous structure.

The coform fibrous structure of the present invention may be made via a suitable coforming process.

“Absorbent structure” as used herein is a structure that when laid out on a horizontal surface defining a plane extending in x- and y-directions, will assume a generally flat 3-dimensional shape having first and second oppositely-facing surfaces that each generally, approximately occupy respective parallel horizontal planes along the x- and y-directions, and having a caliper measured in a z-direction orthogonal to the x- and y-directions; wherein the structure includes one or any combination of fibers, filaments, open-celled foam and particles in an arrangement configured to be capable of receiving, drawing in and holding a quantity of aqueous fluid within the structure. The term includes, by way of example but not limitation, absorbent core structures (or layer components thereof) appearing in products such as diapers, absorbent training pants, adult incontinence pads and pants, and feminine hygiene pads; absorbent household cleaning products, etc. Consistent with the design of the product in which it appears, an “absorbent structure” as defined herein has a fluid receiving side coincident with one of the first and second surfaces, and a non-receiving side opposite the fluid receiving side. In products such as diapers, feminine hygiene pads and other products designed to be worn about the body to absorb bodily exudates, the fluid receiving side of the absorbent structure is proximate the wearer-facing surface of the product, and the non-receiving side is proximate the outward-facing surface of the product.

“Filament” as used herein means an elongate particulate as described above that exhibits a length of greater than or equal to 5.08 cm (2 in.) and/or greater than or equal to 7.62 cm (3 in.) and/or greater than or equal to 10.16 cm (4 in.) and/or greater than or equal to 15.24 cm (6 in.), specifically including all values within these ranges and any ranges created thereby.

Filaments are typically considered continuous or substantially continuous in nature. Filaments are relatively longer than fibers. Non-limiting examples of filaments include meltblown and/or spunbond filaments. Non-limiting examples of polymers that can be spun into filaments include natural polymers, such as starch, starch derivatives, cellulose, such as rayon and/or lyocell, and cellulose derivatives, hemicellulose, hemicellulose derivatives, and synthetic polymers including, but not limited to polyvinyl alcohol filaments and/or polyvinyl alcohol derivative filaments, and thermoplastic polymer filaments, such as polyesters, for example polyethylene terephthalate (PET), nylons, polyolefins such as polypropylene filaments, polyethylene filaments, and polypropylene and polyethylene copolymer filaments, and biodegradable or compostable thermoplastic fibers such as polylactic acid filaments, polyhydroxyalkanoate filaments, polyesteramide filaments, and polycaprolactone filaments. The filaments may be monocomponent or multicomponent, such as bicomponent filaments. In one example, the filaments are monocomponent filaments.

The filaments may be made via spinning, for example via meltblowing and/or spunbonding, from a polymer, for example a thermoplastic polymer, such as polyolefin, for example polypropylene and/or polyethylene, and/or polyester, for example polyethylene terephthalate (PET), and mixtures thereof. Filaments are typically considered continuous or substantially continuous in nature.

The filaments of the present invention may be spun from polymer melt compositions via suitable spinning operations, such as meltblowing and/or spunbonding and/or they may be obtained from natural sources such as vegetative sources, for example trees.

The filaments of the present invention may be monocomponent and/or multicomponent. For example, the filaments may comprise bicomponent fibers and/or filaments. The bicomponent fibers and/or filaments may be in any form, such as side-by-side, core and sheath, islands-in-the-sea and the like.

“Meltblowing” is a process for producing filaments directly from polymers or resins using high-velocity air or another appropriate force to attenuate the filaments before collecting the filaments on a collection device, such as a belt, for example a patterned belt or molding member. In a meltblowing process the attenuation force is applied in the form of high speed air as the material (polymer) exits a die or spinnerette.

“Spunbonding” is a process for producing filaments directly from polymers by allowing the polymer to exit a die or spinnerette and drop a predetermined distance under the forces of flow and gravity and then applying a force via high velocity air or another appropriate source to draw and/or attenuate the polymer into a filament.

“Fiber” as used herein means an elongate particulate as described above that exhibits a length of less than 5.08 cm (2 in.) and/or less than 3.81 cm (1.5 in.) and/or less than 2.54 cm (1 in.), specifically including all values within these ranges and any ranges created thereby. Pulp fibers, for example wood pulp fibers typically exhibit a length of from about 0.7 mm to about 2.5 mm.

Fibers are typically considered discontinuous in nature. Non-limiting examples of fibers include pulp fibers, such as wood pulp fibers, and synthetic staple fibers such as polypropylene, polyethylene, polyester, copolymers thereof, rayon, lyocell, glass fibers and polyvinyl alcohol fibers.

Staple fibers may be produced by spinning a filament tow and then cutting the tow into segments of less than 5.08 cm (2 in.) thus producing fibers; namely, staple fibers.

“Pulp fibers” as used herein means fibers that have been derived from vegetative sources, such as plants and/or trees. In one example of the present invention, “pulp fiber” refers to papermaking fibers. In one example of the present invention, a fiber may be a naturally occurring fiber, which means it is obtained from a naturally occurring source, such as a vegetative source, for example a tree and/or plant, such as trichomes. Such fibers are typically used in papermaking and are oftentimes referred to as papermaking fibers. Papermaking fibers useful in the present invention include cellulosic fibers commonly known as wood pulp fibers. Applicable wood pulps include chemical pulps, such as Kraft, sulfite, and sulfate pulps, as well as mechanical pulps including, for example, groundwood, thermomechanical pulp and chemically modified thermomechanical pulp. Chemical pulps, however, may be preferred since they impart a superior tactile sense of softness to fibrous structures made therefrom. Pulps derived from both deciduous trees (hereinafter, also referred to as “hardwood”) and coniferous trees (hereinafter, also referred to as “softwood”) may be utilized. The hardwood and softwood fibers can be blended, or alternatively, can be deposited in layers to provide a stratified web. Also applicable to the present invention are fibers derived from recycled paper, which may contain any or all of the above categories of fibers as well as other non-fibrous polymers such as fillers, softening agents, wet and dry strength agents, and adhesives used to facilitate the original papermaking.

In one example, the wood pulp fibers are selected from the group consisting of hardwood pulp fibers, softwood pulp fibers, and mixtures thereof. The hardwood pulp fibers may be selected from the group consisting of: tropical hardwood pulp fibers, northern hardwood pulp fibers, and mixtures thereof. The tropical hardwood pulp fibers may be selected from the group consisting of: eucalyptus fibers, acacia fibers, and mixtures thereof. The northern hardwood pulp fibers may be selected from the group consisting of: cedar fibers, maple fibers, and mixtures thereof.

In addition to the various wood pulp fibers, other natural/naturally-occurring cellulose fibers such as cotton fibers, cotton linters, rayon, lyocell, trichomes, seed hairs, rice straw, wheat straw, bamboo, and bagasse fibers can be used in this invention. Other sources of cellulose in the form of fibers or capable of being spun into fibers include grasses and grain sources.

“Trichome” or “trichome fiber” as used herein means an epidermal attachment of a varying shape, structure and/or function of a non-seed portion of a plant. In one example, a trichome is an outgrowth of the epidermis of a non-seed portion of a plant. The outgrowth may extend from an epidermal cell. In one embodiment, the outgrowth is a trichome fiber. The outgrowth may be a hairlike or bristle like outgrowth from the epidermis of a plant.

Trichome fibers are different from seed hair fibers in that they are not attached to seed portions of a plant. For example, trichome fibers, unlike seed hair fibers, are not attached to a seed or a seed pod epidermis. Cotton, kapok, milkweed, and coconut coir are non-limiting examples of seed hair fibers.

Further, trichome fibers are different from non-wood bast and/or core fibers in that they are not attached to the bast, also known as phloem, or the core, also known as xylem portions of a non-wood dicotyledonous plant stem. Non-limiting examples of plants which have been used to yield non-wood bast fibers and/or non-wood core fibers include kenaf, jute, flax, ramie and hemp.

Further trichome fibers are different from monocotyledonous plant derived fibers such as those derived from cereal straws (wheat, rye, barley, oat, etc.), stalks (corn, cotton, sorghum, Hesperaloe funifera, etc.), canes (bamboo, bagasse, etc.), grasses (esparto, lemon, sabai, switchgrass, etc.), since such monocotyledonous plant derived fibers are not attached to an epidermis of a plant.

Further, trichome fibers are different from leaf fibers in that they do not originate from within the leaf structure. Sisal and abaca are sometimes liberated as leaf fibers.

Finally, trichome fibers are different from wood pulp fibers since wood pulp fibers are not outgrowths from the epidermis of a plant; namely, a tree. Wood pulp fibers rather originate from the secondary xylem portion of the tree stem.

The fibrous structures of the present invention may be homogeneous or may be layered. If layered, the fibrous structures may comprise at least two and/or at least three and/or at least four and/or at least five layer of fiber and/or filament compositions. In one example, the fibrous structure of the present invention consists essentially of fibers, for example pulp fibers, such as cellulosic pulp fibers and more particularly wood pulp fibers.

In another example, the fibrous structures of the present invention comprise fibers and are void of filaments.

In still another example, the fibrous structures of the present invention comprise filaments and fibers, such as a coform fibrous structure.

“Coform fibrous structure” or “coformed fibrous structure” and/or “coform absorbent core” as used herein means that the fibrous structure comprises a mixture of at least two different materials wherein at least one of the materials comprises a filament, such as a polypropylene filament and/or polylactic acid filament, and at least one other material, different from the first filament material mentioned above, comprises a solid additive, such as a fiber and/or a particulate, for example a pulp fiber. In one example, a coform fibrous structure comprises solid additives, such as fibers, for example pulp fibers, such as wood pulp fibers, and filaments, such as polypropylene filaments. “Basis Weight” as used herein is the weight per unit area of a sample reported in lbs./3000 ft² or g/m² (gsm) and is measured according to the Basis Weight Test Method described herein.

“Machine Direction” or “MD” as used herein means the direction parallel to the flow of the fibrous structure through the fibrous structure making machine and/or sanitary tissue product manufacturing equipment. This axis is also commonly considered the Y or longitudinal axis as shown in the drawings.

“Cross Machine Direction” or “CD” as used herein means the direction parallel to the width of the fibrous structure making machine and/or sanitary tissue product manufacturing equipment and perpendicular to the machine direction. This axis is also commonly considered the X or lateral axis as shown in the drawings.

“Different” as used herein with respect to particles, means two or more particles exhibit different properties for example different sizes, shapes, densities, masses, Stokes Numbers, and/or compositions.

“Ply” as used herein means an individual, integral fibrous structure.

“Plies” as used herein means two or more individual, integral fibrous structures disposed in a substantially contiguous, face-to-face relationship with one another, forming a multi-ply sanitary tissue product. It is also contemplated that an individual, integral fibrous structure can effectively form a multi-ply sanitary tissue product, for example, by being folded on itself.

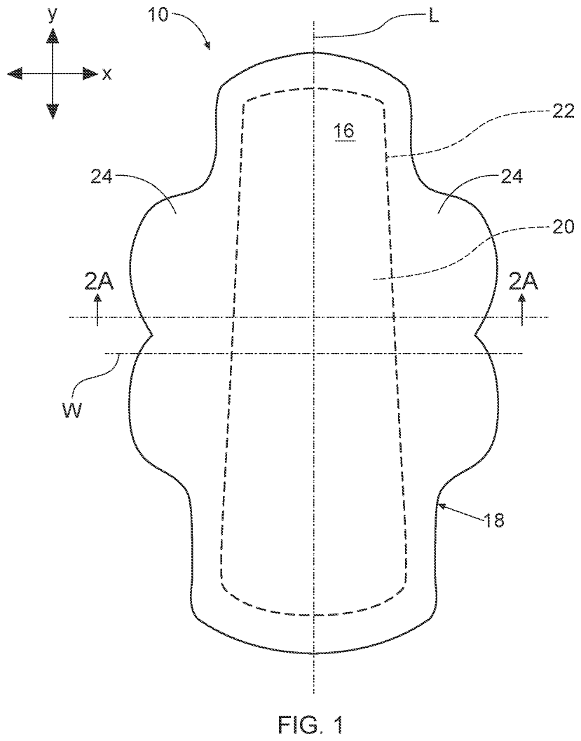

“X” (for example the lateral axis in FIG. 1) or “Y” (for example the longitudinal axis in FIG. 1) or “xy”, and “Z” (for example the axis orthogonal to the lateral axis and longitudinal axis in FIG. 1) or “z” designate a conventional system of Cartesian coordinates, wherein mutually perpendicular coordinates “X” and “Y” define a reference X-Y (xy) plane, and “Z” defines an orthogonal to the X-Y plane. “Z-direction” designates any direction perpendicular to the X-Y plane. Analogously, the term “Z-dimension” means a dimension, distance, or parameter measured parallel to the Z-direction. When an element, such as, for example, a molding member curves or otherwise deplanes, the X-Y plane follows the configuration of the element.

With respect to a continuous manufacturing line, the terms “upstream” and “downstream” are relative terms that positionally relate locations of items of equipment and/or processes on the line, relative a general path of movement of materials and/or products along the line. Materials and/or products move along the line from an upstream location to a downstream location.

“Non-elastic” as used herein means a material does not exhibit elastic properties and/or elasticity and/or elastomeric.

As used herein, the articles “a” and “an” when used herein, for example, “an anionic surfactant” or “a fiber” is understood to mean one or more of the material that is claimed or described.

All percentages and ratios are calculated by weight unless otherwise indicated. All percentages and ratios are calculated based on the total composition unless otherwise indicated.

Unless otherwise noted, all component or composition levels are in reference to the active level of that component or composition, and are exclusive of impurities, for example, residual solvents or by-products, which may be present in commercially available sources.

### Absorbent Article

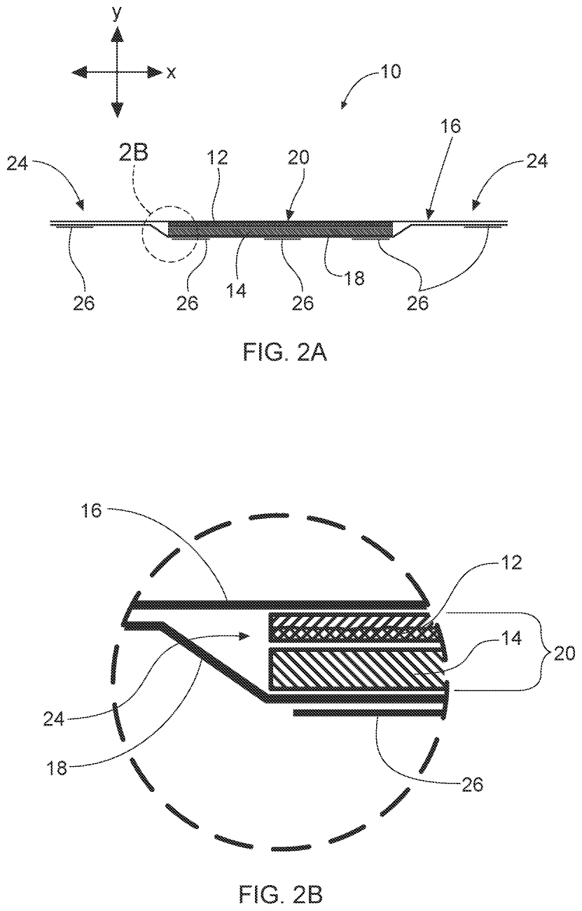

As shown in FIGS. 1, 2A and 2B, an absorbent article 10 of the present invention, a non-limiting example of which is a feminine hygiene pad, comprises a layered fluid acquisition/distribution system 12, for example a secondary topsheet, and optionally one or more additional fluid handling systems, such as a fluid storage system 14, for example an absorbent core, and/or a topsheet 16 and/or a backsheet 18. Consistent with the design of the absorbent article product type in which it appears, a fluid handling system, for example a layered fluid acquisition/distribution system 12 and/or a fluid storage system 14, when present, as described herein has a fluid receiving side that will encounter fluid to be managed, for example acquired, transferred, distributed, captured and/or absorbed, immediately or shortly after discharge or other exposure thereto, and an oppositely-disposed non-fluid-receiving side that will in use face away from the discharge or other exposure of fluid. By way of a non-limiting example, in absorbent articles such as diapers, feminine hygiene pads and other absorbent articles designed to be worn on and/or about the body to manage bodily exudates, the fluid receiving side of the fluid handling system is proximate the wearer-facing surface of the product, and the non-fluid-receiving side of the fluid handling system is proximate the outward-facing surface of the absorbent article.

In one example, the components (topsheet, and layered fluid acquisition/distribution system and fluid storage system (when present)) are arranged and oriented within the absorbent article such that a capillarity cascade is created such that fluid moves from the topsheet, (the component that receives initial fluid insult) with a capillarity that is less than the capillarity of the layered fluid acquisition/distribution system to the layered fluid acquisition/distribution system with a capillarity that is higher than, for example substantially higher than, the capillarity of the topsheet. In cases where the absorbent article comprises a fluid storage system, in one example, the capillarity cascade is such that the fluid moves from the layered fluid acquisition/distribution system with a capillarity that is less than the capillarity of the fluid storage system to the fluid storage system with a capillarity that is higher than, for example substantially higher than, the capillarity of the layered fluid acquisition/distribution system. In one example, the capillary pressure of the layered fluid acquisition/distribution system is greater than the capillary pressure of the topsheet. In cases where the absorbent article comprises a fluid storage system, in one example, the capillary pressure of the fluid storage system is greater than the capillary pressure of the layered fluid acquisition/distribution system. In one example, the base nonwoven substrate and/or the coform fibrous structure of the layered fluid acquisition/distribution system exhibit capillarity and/or capillary pressures that do not negatively impact the capillarity cascade described above.

In one example, the base nonwoven substrate and/or the coform fibrous structure of the layered fluid acquisition/distribution system exhibit sufficient wettability and/or liquid permeability so that fluid readily enters the base nonwoven substrate of the layered fluid acquisition/distribution system and be partitioned to the coform fibrous structure of the layered fluid acquisition/distribution system to enable good masking and/or dryness there the absorbent article contacts a users' body.

In one example, the base nonwoven substrate and/or the coform fibrous structure of the layered fluid acquisition/distribution system comprise fibrous elements, for example fibers and/or filaments, that are flexible, conformable and/or not too stiff or too rough to the touch.

In one example, the base nonwoven substrate and/or the coform fibrous structure of the layered fluid acquisition/distribution system comprise fibrous elements, for example fibers and/or filaments, that comprise synthetic polymers, natural polymers, and/or sustainable, such as biodegradable and/or compostable, polymers.

In one example, the base nonwoven substrate of the layered fluid acquisition/distribution system exhibits sufficient air permeability to effectively permit the deposition and/or collecting of the coform fibrous structure on a surface of the base nonwoven substrate such that a resulting layered fluid acquisition/distribution system of the present invention is formed.

In one example, the base nonwoven substrate and/or the coform fibrous structure of the layered fluid acquisition/distribution system exhibit sufficient tensile strength to survive MD and/or CD forces experienced by the base nonwoven substrate and/or the coform fibrous structure and/or the layered fluid acquisition/distribution system during processing steps, for example assembling, converting, and/or splicing, to form an absorbent article of the present invention.

For example, to move a fluid the capillarity force necessary to move the fluid from a topsheet to a layered fluid acquisition/distribution system (secondary topsheet), the layered fluid acquisition/distribution system's capillarity needs to be substantially higher than the topsheet's capillarity.

As shown in FIGS. 1, 2A and 2B, an absorbent article 10 of the present invention, for example feminine hygiene pad, comprises a layered fluid acquisition/distribution system 12, for example a unitary, layered fluid acquisition/distribution system, such as a layered liquid acquisition/distribution system, according to the present invention. In addition, the absorbent article 10 of the present invention may further comprise a topsheet 16, for example a fluid permeable, such as a liquid permeable topsheet, a backsheet 18, for example a fluid impermeable, such as a liquid impermeable backsheet, and a fluid storage system 14, for example a liquid storage system, such as an absorbent core. As shown in FIGS. 1, 2A and 2B, the layered fluid acquisition/distribution system 12 may be disposed between the topsheet 16 and the backsheet 18, when both the topsheet 16 and backsheet 18 are present in the absorbent article 10. In another example, the layered fluid acquisition/distribution system 12 may be disposed between the topsheet 16 and the fluid storage system 14, when both the topsheet 16 and fluid storage system 14 are present in the absorbent article 10. In another example, the fluid storage system 14 may be disposed between the layered fluid acquisition/distribution system 12 and the backsheet 18, when both the backsheet 18 and fluid storage system 14 are present in the absorbent article 10.

In one example, as shown in FIGS. 2A and 2B, the layered fluid acquisition/distribution system 12 of the absorbent article 10 may be associated, with or without bonding, for example bonding by adhesive bonding and/or mechanical bonding, with the fluid storage system 14, to form an absorbent core structure 20. The absorbent core structure 20 and/or the layered fluid acquisition/distribution system 12 may exhibit an outer perimeter edge 22. In regions outside the outer perimeter edge 22, the topsheet 16 and the backsheet 18, when both are present, may be bonded together in laminated fashion by any suitable mechanism including but not limited to adhesive bonding, thermal bonding, pressure bonding, etc., thereby enveloping the absorbent core structure, when both the layered fluid acquisition/distribution system 12 and the fluid storage system 14 are present, or the layered fluid acquisition/distribution system 12 by itself when a fluid storage system 14 is not present.

In one example, as shown in FIGS. 1, 2A and 2B, an absorbent article 10 of the present invention may optionally comprise opposing wing portions 24 extending laterally outside of outer perimeter edge 22 by a comparatively greater width dimension than the main portion of the absorbent article 10. The outer surface of the backsheet 18 forming the undersides of the main portion and the wing portions 24 may have deposits of adhesive 26 thereon. Adhesive deposits 26 may be provided to enable the user to adhere the absorbent article 10 to the inside of a user's underpants in the crotch region thereof, and wrap the wing portions 24 through and around the inside edges of the leg openings of the underpants and adhere them to the outside/underside of the underpants in the crotch region, providing supplemental holding support and helping protect the leg edges of the underpants from soiling by exudates. When absorbent article 10 is packaged, adhesive deposits 26 may be covered by one or more sheets of release film or paper (not shown) that covers/shields the adhesive deposits 26 from contact with other surfaces until the user is ready to remove the release film or paper and place the absorbent article 10 in the underpants for use.

With respect to components of an absorbent article 10 configured to be worn on the body for purposes of absorbing bodily exudates, as shown in FIG. 1, the relative terms “top” and “upper” refer to portions more proximate the wearer-facing surface of the absorbent article 10, and the relative terms “bottom” and “lower” refer to portions more proximate the outward-facing surface of the absorbent article 10. Similarly, when a first component or feature is said to be “above” a second component or feature, the first component or feature is disposed relatively closer to the wearer-facing surface of the absorbent article 10 than the second component or feature; and the second component or feature is “below” the first component or feature. Similarly, when a first component or feature is said to be “superadjacent” a second component or feature, the first component or feature is disposed immediately above the second component or feature (with no components or features between them); and the second component or feature is “subjacent” the first component or feature.

With respect to an absorbent article 10 as shown in FIG. 1, such as a feminine hygiene pad, in a flattened and unfolded condition resting on a horizontal planar surface with major macroscopic surfaces of its major constituent components in a flattened and unfolded condition occupying planes substantially parallel the planar surface, the x- and y-directions are 90° from one another and oriented parallel to the planar surface, and the z-direction is orthogonal to the x- and y-directions, perpendicular to the planar surface. For purposes herein the y-direction, of an absorbent article 10, such as a feminine hygiene pad, configured to be worn about a user's lower torso through the user's crotch area, will be parallel the longitudinal direction as defined herein; the x-direction will be parallel the lateral direction as defined herein; and the z-direction will be orthogonal to the longitudinal and lateral directions as defined herein. The caliper of an absorbent article 10 or component thereof is measured along the z-direction.

With respect to an absorbent article 10 as shown in FIG. 1, such as a feminine hygiene pad, configured to be worn about a user's lower torso through the user's crotch area, that is opened and laid out flat on a horizontal planar surface, “lateral” refers to a direction perpendicular to the longitudinal direction and parallel the horizontal planar surface. “Width” W refers to a dimension measured in the lateral direction.

With respect to an absorbent article 10 as shown in FIG. 1, such as a feminine hygiene pad, configured to be worn about a user's lower torso through the user's crotch area, that is opened and laid out flat on a horizontal planar surface and having a length measured from its forwardmost end to its rearwardmost end (as the product would be worn used normally by a user), “longitudinal” refers to a direction parallel with the line along which the length is measured, and parallel to the horizontal planar surface. “Length” L refers to a dimension measured in the longitudinal direction.

With respect to an absorbent article 10 as shown in FIG. 1, such as a feminine hygiene pad, configured to be worn about a user's lower torso through the user's crotch area, the terms “front,” “rear,” “forward” and “rearward” relate to features or regions of the absorbent article 10 corresponding to the position it would occupy as normally worn by a user, with respect to the front and rear of the user's body when standing.

When an absorbent article 10 as shown in FIG. 1, such as a feminine hygiene pad, configured to be worn about a user's lower torso through the user's crotch area, is being worn by a user (and thus has been urged into a curved configuration), “z-direction” at any particular point location on the absorbent article 10 refers to a direction normal to the wearer-facing surface of the absorbent article 10 at the particular point location. With respect to a web material, for example a fibrous structure, during its manufacture or processing, “z-direction” refers to a direction orthogonal to both the machine direction and the cross direction of manufacture or processing, and any plane parallel to the machine direction and cross direction may be referred to as an “x-y plane”.

In one example, an absorbent article of the present invention exhibits a basis weight of from greater than 50 gsm and/or greater than 100 gsm and/or greater than 150 gsm and/or greater than 200 gsm and/or to less than 1000 gsm and/or to less than 800 gsm and/or to less than 600 gsm and/or to less than 500 gsm and/or to less than 400 gsm, specifically including all values within these ranges and any ranges created thereby, as measured according to the Basis Weight Test Method described herein.

### Layered Fluid Acquisition/Distribution System

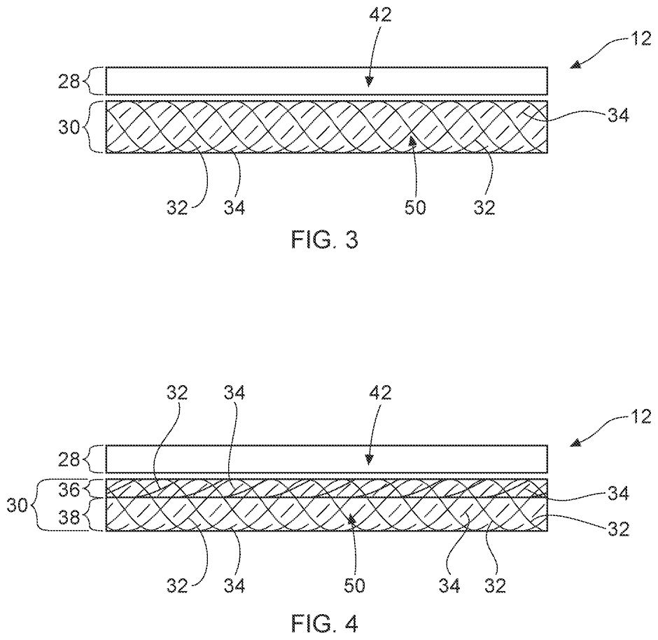

As shown in FIG. 3, a layered fluid acquisition/distribution system 12 of the present invention, for example a unitary, layered fluid acquisition/distribution system, comprises a first layer 28 comprising a base nonwoven substrate 42, such as a carded base nonwoven substrate and/or a spunlace base nonwoven substrate and/or a hydroentangled base nonwoven substrate and/or a spunbond base nonwoven substrate, and a second layer 30, comprising a mixture of a plurality of fibrous elements, for example a plurality of filaments 32, for example thermoplastic filaments, which may be biodegradable thermoplastic filaments and/or compostable thermoplastic filaments, and a plurality of fibers 34, for example pulp fibers, such as a coform fibrous structure 50.

The layered fluid acquisition/distribution system may be separate and apart from the other fluid handling systems present in the absorbent article.

In one example, the layered fluid acquisition/distribution system may be associated with, for example bonded to, such as thermally bonded to and/or adhesively bonded to and/or chemically bonded to, one or more other fluid handling systems, such as a fluid storage system, for example an absorbent core to form an absorbent core structure, present in the absorbent article.

As shown in FIG. 4, a layered fluid acquisition/distribution system 12 of the present invention, for example a unitary, layered fluid acquisition/distribution system comprises a first layer 28 comprising a base nonwoven substrate 42, such as a carded base nonwoven substrate and/or a spunlace base nonwoven substrate and/or a hydroentangled base nonwoven substrate and/or a spunbond base nonwoven substrate, and a second layer 30, comprising a mixture of a plurality of fibrous elements, for example a plurality of filaments 32, for example thermoplastic filaments, which may be biodegradable thermoplastic filaments and/or compostable thermoplastic filaments, and a plurality of fibers 34, for example pulp fibers, such as a coform fibrous structure 50, which in this case is a layered coform fibrous structure. In one example, the second layer 30, for example the coform fibrous structure 50, which in this case is a layered coform fibrous structure, comprises two or more sublayers 36, 38. In one example, sublayer 36 comprises relatively larger average diameter filaments 32, for example average diameter filaments of from about 12 to about 20 μm, compared to sublayer 38 which comprises relatively smaller average diameter filaments 32, for example average diameter filaments of from about 3 to about 10 μm. In one example, sublayer 36 comprises relatively larger average diameter fibers 34 compared to sublayer 38 which comprises relatively smaller average diameter fibers 34. In one example, sublayer 36 comprises relatively larger average diameter filaments 32 and relatively larger average diameter fibers 34 compared to sublayer 38 which comprises relatively smaller average diameter filaments 32 and relatively smaller average diameter fibers 34. In one example, sublayer 36 exhibits a relatively lower density (for example more open and/or more lofty structure) to permit fluids to more easily pass through compared to sublayer 38 which exhibits a relatively higher density (for example less open and/or less lofty structure) such that the second layer 30 exhibits a capillarity gradient with sublayer 36 exhibiting a lower capillarity than sublayer 38. In another example, sublayer 36 functions as an acquisition layer receiving fluid from the first layer 28 and then sublayer 38 functions both as an acquisition layer receiving fluid from sublayer 36 and as a distribution layer distributing the fluid along an interface between it and another fluid handling system, such as a fluid storage system, and/or optionally storing the fluid. In one example, the capillarity of sublayer 38 needs to be higher, for example substantially higher than the capillarity of sublayer 36, which in turn needs to be higher, for example substantially higher than the capillarity of the first layer 28.

In one example, a layered fluid acquisition/distribution system of the present invention may have a basis weight from about 30 gsm to about 100 gsm, from about 40 gsm to about 75 gsm, or from about 50 gsm to about 60 gsm, specifically including all values within these ranges and any ranges created thereby, as measured according to the Basis Weight Test Method described herein.

In one example, the layered fluid acquisition/distribution system of the present invention is configured to wick fluid from the primary location of discharge of fluid onto the absorbent article, along any or all of the x-, y-, and z-directions, thereby distributing the fluid to and along an underlying fluid storage system, for example an absorbent core, when present.

The layered fluid acquisition/distribution system of the present invention may comprise two or more layers of materials. In one example, the layered fluid acquisition/distribution system comprises a first layer comprising a base nonwoven substrate, for example a fibrous structure comprising fibers, for example a carded base nonwoven substrate comprising a plurality of fibers, such as synthetic fibers, for example thermoplastic polymer fibers and/or synthetic staple fibers, polyolefin fibers, such as polypropylene fibers, polyester fibers, such as polyethylene terephthalate fibers (PET), and/or bicomponent fibers, such as polyethylene/polypropylene (PE/PP) bicomponent fibers and/or polyethylene/polyethylene terephthalate (PE/PET) bicomponent fibers and blends thereof, cellulosic fibers, such as regenerated cellulose fibers, for example rayon fibers, viscose fibers and/or lyocell fibers, and/or cotton fibers and/or hemp fibers and/or a fibrous structure comprising blends and/or mixtures of both sustainable synthetic fibers, for example biodegradable and/or compostable fibers and synthetic fibers non-limiting examples of which are described above, with or without chemistries such as wetting agents, emollients, clean chemistries, for example antifouling chemistries, and the like. In another example, the first layer may comprise sub-layers, for example a first sub-layer comprising a carded base nonwoven substrate comprising a plurality of fibers, such as pulp fibers, for example wood pulp fibers, and a second sub-layer associated, integrated with the first sub-layer, for example by depositing the second sub-layer materials on a surface of the first sub-layer, wherein the second sub-layer comprises an airlaid mixture of fibers, for example pulp fibers, such as wood pulp fibers, and a plurality of synthetic fibers, such as synthetic staple fibers, for example bicomponent staple fibers, such as thermoplastic bicomponent staple fibers.

The second layer of the layered fluid acquisition/distribution system comprises a plurality of filaments and a plurality of fibers, for example in the form of a coform fibrous structure. In one example, the second layer comprises a coform fibrous structure. The coform fibrous structure may be a homogeneous coform fibrous structure or a layered coform fibrous structure comprising two or more sub-layers of filaments and fibers that are different from each other, for example differ in average filament diameter, fiber types, filament materials, density, capillarity, and combinations thereof.

In one example, the second layer is associated with, for example deposited on, such as filaments of the second layer are spun from a die, mixed with fibers and then laid directly on a surface of the first layer.

The fibers of the fluid acquisition/distribution system may comprise pulp fibers. The pulp fibers may be selected from the group consisting of wood pulp fibers, non-wood pulp fibers and mixtures thereof.

The fibers and/or filaments of the fluid acquisition/distribution system may comprise synthetic fibers. The synthetic fibers and/or filaments may comprise thermoplastic polymers, for example polyolefins, polyesters, polycaprolactones, polylactic acids, polyhydoxyalkanoates, and mixtures thereof. The synthetic fibers and/or filaments may comprise biodegradable polymers, compostable polymers and mixtures thereof. The synthetic fibers and/or filaments may comprise recycled and/or recyclable polymers.

In one example, one or more layers of the fluid acquisition/distribution system may comprise a homogenous mix of fibers.

In another example, one or more layers of the fluid acquisition/distribution system may comprise a heterogeneous mix of fibers. For example, typically a plurality of carding machines feed a spunlace process. The types of fibers supplied to the cards may be homogeneously blended as mentioned above. Or in contrast, the types of fibers or the weight percentage of the fibers provided to the carding machines may be different. In such forms, where the types of fibers and/or the weight percentage of the fibers are varied to the carding machines, the resulting spunlaced structure may comprise a plurality of heterogeneous strata which areafter the spunlacing process integral with one another.

A layered fluid acquisition/distribution system may be formed by a top layer performing the function of high masking, fast acquiring yet efficient draining with high wet compressive recovery as measured according to the Wet and Dry Bunched Compression Test Method described herein, integral with a coform (commingled mixture of filaments and fibers, for example pulp fibers) distribution layer whereby the integrated absorbent element, for example the fibers, such as pulp fibers, provides both the needed fluid absorption and distribution function while being soft, cushy and conformable yet recoverable from in-use compressive forces whether dry or loaded.

### 1. First Layer-Base Nonwoven Substrate

In one example, the first layer comprises a base nonwoven substrate, such as a carded base nonwoven substrate and/or a spunlace base nonwoven substrate and/or a hydroentangled base nonwoven substrate and/or a spunbond base nonwoven substrate. The base nonwoven substrate may exhibit a basis weight from about 10 gsm to about 60 gsm and/or from about 10 gsm to about 50 gsm and/or from about 20 to about 50 gsm and/or from about 20 gsm to about 40 gsm, specifically including all values within these ranges and any ranges created thereby, as measured according to the Basis Weight Test Method described herein.

In one example, the base nonwoven substrate may comprise a homogeneous mix of fibers.

In one example, the base nonwoven substrate may comprise a heterogeneous mix of fibers. For example, typically a plurality of carding machines feed a spunlace process. The types of fibers supplied to the cards may be homogeneously blended as mentioned above. Or in contrast, the types of fibers or the weight percentage of the fibers provided to the carding machines may be different. In such forms, where the types of fibers and/or the weight percentage of the fibers are varied to the carding machines, the resulting spunlaced structure may comprise a plurality of heterogeneous strata which are—after the spunlacing process—integral with one another.

For those forms where the base nonwoven substrate comprises a plurality of heterogeneous strata, an acquisition gradient may be achieved with careful selection of the fibers within each of the stratum of the base nonwoven substrate. For example, a first stratum-being closest in proximity to the topsheet, when present, —may include a lower amount of absorbent fiber as opposed to a stratum which is disposed further from the topsheet. In one example, the first stratum may include from between about 35% by weight to about 90% nonabsorbent fibers while a stratum disposed furthest from the topsheet may include about 15% by weight to about 30% by weight absorbent fiber. In one example, the level of any stiffening fibers within the base nonwoven substrate may stay constant among the strata or may be varied to create a stiffness gradient in the acquisition layer in addition to the absorbency gradient. Similarly, in one example, the level of any resilient fibers may stay constant among the strata or may be varied to create a permeability or capillarity gradient in the acquisition layer in addition to the absorbency gradient or in addition to the stiffness gradient. Forms are contemplated where the base nonwoven substrate comprises between 1 to 4 strata.

Some exemplary fibers that may be included in the base nonwoven substrate may include absorbent fibers, stiffening fibers, and resilient fibers. Forms are contemplated where at least one of the absorbent fibers, stiffening fibers, and/or resilient fibers comprise a hydrophilic coating. Suitable hydrophilic coatings are known in the art. Additionally, in some forms, the one or more of the above fibers of the base nonwoven substrate may comprise a staple length, for example about 38 mm or 50 mm.

Any suitable absorbent fibers may be utilized. Conventional absorbent fibers include cotton, rayon or regenerated cellulose. In one example, the base nonwoven substrate may comprise viscose fibers. Due to the proximity of the base nonwoven substrate to the topsheet, the absorbent fibers can help to draw discharged fluid from the topsheet into the layered fluid acquisition/distribution system. In one example, the base nonwoven substrate may comprise from about 20% by weight to about 50% by weight and/or from about 21% by weight to about 40% by weight and/or from about 25% by weight to about 30% by weight, specifically including any values within these ranges and any ranges created thereby, of absorbent fibers. In one example, the base nonwoven substrate may comprise about 25% by weight absorbent fibers.

A higher weight percentage of absorbent fibers may be beneficial for fluid insults that are more viscous, for example menstrual fluid. However, the inclusion of a higher weight percentage of absorbent fibers can negatively impact resiliency and stiffness of the base nonwoven substrate. And, too low of a weight percentage of absorbent fibers can result in a more ‘wet feeling’ topsheet which can create a negative impression of the product in consumers' minds. The weight percentages provided above may also work well in the context of urinary fluid insults.

Any suitable size of absorbent fiber may be utilized. A suitable measure of size can be linked to linear density. In some forms, the absorbent fiber linear density may range from about 2 dtex to about 6 dtex, about 2.5 dtex to about 4 dtex, or from about 2.8 dtex to about 3.5 dtex, specifically reciting all values within these ranges and any ranges created thereby. In one specific form, the absorbent fiber may comprise a linear density of about 3.3 dtex.

The absorbent fibers may have any suitable shape. In some forms, a trilobal shape may be utilized. The trilobal shape can improve wicking and improve masking. Trilobal rayon is available from Kelheim Fibres and sold under the trade name Galaxy.

In addition to absorbent fibers, as mentioned previously, the base nonwoven substrate may also comprise stiffening fibers. Stiffening fibers may be utilized to help provide structural integrity to the base nonwoven substrate. The stiffening fibers can help increase structural integrity of the base nonwoven substrate in a machine direction and in a cross machine direction which facilitate web manipulation during processing of the base nonwoven substrate for incorporation into the layered fluid acquisition/distribution system and/or an absorbent article. In one example, the base nonwoven substrate may be heat stiffened. The heat stiffening process can create a plurality of connection points amongst the stiffening fibers. In general, the higher the number of connection points, the stiffer the base nonwoven substrate. So, while the creation of a plurality of connection points is beneficial for processability, the creation of too many connection points can lead to a base nonwoven substrate which is uncomfortable in its respective absorbent article. With that in mind, the constituent material of the stiffening fibers, the weight percentage of the stiffening fibers, and heat of processing should be carefully selected. The heat stiffening process is discussed hereafter.

With the foregoing in mind, any suitable stiffening fiber may be utilized. Some examples of suitable stiffening fibers include bi-component fibers comprising polyethylene and polyethylene terephthalate components or polyethylene terephthalate and co-polyethylene terephthalate components. The components of the bi-component fiber may be arranged in a core sheath arrangement, a side by side arrangement, an eccentric core sheath arrangement, a trilobal arrangement, or the like. In one specific example, the stiffening fibers may comprise bi-component fibers having polyethylene/polyethylene terephthalate components arranged in a concentric, core—sheath arrangement where the polyethylene is the sheath. In some forms, mono-component fibers may be utilized. In such forms, the constituent material of the mono-component may comprise polypropylene.

Any suitable size of stiffening fiber may be utilized. Suitable linear densities of stiffening fiber may be from about 4 dtex to about 12 dtex, from about 4.5 dtex to about 10 dtex, or from about 5 dtex to about 7 dtex, specifically reciting all values within these ranges and any ranges created thereby. In one specific form, the stiffening fibers may comprise a linear density of about 5.8 dtex polyethylene/polyethylene terephthalate bi-component fibers arranged in a core and concentric sheath arrangement.

Any suitable weight percentage of stiffening fibers may be utilized in the base nonwoven substrate as well. However, in some forms, the base nonwoven substrate may be heat treated (heat stiffened). The heat treatment can create connection points amongst the fibers of the base nonwoven substrate. So, where there is a higher percentage of stiffening fibers, more connection points may be created. The additional connection point can yield a much stiffer base nonwoven substrate which may negatively impact comfort. In one example, the base nonwoven substrate may comprise about 20% to about 40% by weight stiffening fibers and/or from about 25% to about 35% by weight stiffening fibers, specifically including all values within these ranges and any ranges created thereby.

As noted previously, the base nonwoven substrate may additionally comprise resilient fibers. The resilient fibers can help the base nonwoven substrate maintain its permeability. Any suitable size fiber may be utilized. In one example, the resilient fibers can have a linear density of about 4 dtex to about 12 dtex and/or from about 5 dtex to about 10 dtex and/or from about 6 dtex to about 8 dtex, specifically reciting all values within these ranges and any ranges created thereby. In one example, the resilient fibers may have a linear density of about 10 dtex. In one example, the resilient fibers may comprise hollow spiral polyethylene terephthalate fibers having a linear density of about 10 dtex.

If smaller fiber sizes are utilized, the resiliency of the base nonwoven substrate would be expected to decrease. And, with the decreased size at the same weight percentage, a higher number of fibers per gram would equate to a decrease in permeability (or increase in capillarity) of the base nonwoven substrate. Additionally, some conventional nonwoven substrates may utilize superabsorbent polymer, for example AGM, to help drain their respective topsheets.

Any suitable weight percentage of resilient fibers may be utilized. Resilient fibers as used herein means that the fibers are able to deform and recover under mechanical stress of up to about 10 kPa. In one example, the base nonwoven substrate may comprise from about 20% to about 70% by weight resilient fibers and/or between 35% and 50% and/or between 40% and 45% by weight resilient fibers, specifically including any values within these ranges and any ranges created thereby. In one example, the base nonwoven substrate may comprise about 45% by weight resilient fibers. In one example, the base nonwoven substrate may comprise about 45% by weight of hollow spiral polyethylene terephthalate fibers having a linear density of about 10 dtex.

With regard to the heat stiffening process, any suitable temperature may be utilized provided it does not physically damage (such as completely melt or burn) them. And, the suitable temperature may be impacted, in part, by the constituent chemistry of the stiffening fibers as well as by processing speed of the base nonwoven substrate. In some forms, the base nonwoven substrate may be heat stiffened at a temperature of 132° C. Additionally, in order to provide a uniform stiffness property across the base nonwoven substrate, any heating operation should be set up to provide uniform heating to the base nonwoven substrate. Even small variations in temperature can greatly impact the tensile strength of the base nonwoven substrate. For example, for two comparable base nonwoven substrates having a basis weight of about 50 gsm, both with the above formulations, a significant difference was created with a small temperature difference. A heat stiffening process at 135° C. yielded a CD direction tensile strength for one sample that was twice the CD direction tensile strength of a sample subjected to a 132° C. stiffening process. A similar result was witnessed for samples having comparable compositions and about a 70 gsm basis weight. Additionally, there was about a 1.5 times difference for the MD direction tensile strength where the sample subjected to the higher temperature, i.e., 135° C., had a higher tensile strength in the MD direction.

In one example, the base nonwoven substrate comprises a lofty carded base nonwoven substrate, for example a lofty carded base nonwoven substrate comprising polyethylene/polyethylene terephthalate bicomponent filaments, such as a 24 gsm 100% PE/PET Bico, having a linear density of about 2.0 dtex fibers commercially available under the tradename Aura 20 from Xiamen Yanjan New Material Co., China.

In one example, the base nonwoven substrate exhibits a caliper of from about 0.2 mm to about 1 mm and/or from about 0.2 mm to about 0.75 mm and/or from about 0.3 mm to about 0.75 mm as measured according to the Caliper Test Method described herein.

In one example, the base nonwoven substrate exhibits an air permeability of from about 150 m³/m²/min and about 500 m³/m²/min according to Air Permeability Test Method described herein.

In one example, the base nonwoven substrate is porous to allow sufficient airflow for the coform fibrous structure to be deposited and/or integrated into the base nonwoven substrate to form the layered fluid acquisition/distribution system of the present invention.

In one example, the average diameter of the fibers of the base nonwoven substrate is greater than about 10 μm and/or greater than about 15 μm to about 50 μm and/or to about 30 μm and/or from about 10 μm to about 50 μm and/or from about 15 μm to about 30 μm as measured according to the Average Diameter Test Method described herein.

In one example, the linear densities of the fibers of the base nonwoven substrates are from about 1.5 dtex to about 10 dtex and/or from about 2 dtex to about 6 dtex for homogeneous base nonwoven substrates, such as carded base nonwoven substrates, for example air through bonded carded base nonwoven substrates, spunbond base nonwoven substrates, and spunlace base nonwoven substrates.

In one example, the linear densities of the fibers of the base nonwoven substrates are from about 1.5 dtex to about 10 dtex and/or from about 2 dtex to about 6 dtex for heterogeneous base nonwoven substrates, for example base nonwoven substrates that comprise a blend and/or mixture of different fibers and/or fiber types and/or fibers that exhibit different linear densities, such as hydroentangled base nonwoven substrates, for example a hydroentangled base nonwoven substrate comprising a blend and/or mixture of fibers, for example synthetic fibers, such as synthetic staple fibers, comprising up to 30% by weight of rayon fibers having a linear density of from about 1.3 dtex to about 2 dtex, up to 70% by weight of polyolefin, for example polypropylene, polyester, for example polyethyleneterephthalate (PET), and/or bicomponent fibers, for example polyethylene/polypropylene core/sheath fibers and/or polyethylene/polyester fibers, for example PE/PET fibers, having a linear density of from about 2 dtex to about 6 dtex, and up to 30% bicomponent fibers, for example polyethylene/polyester fibers, for example PE/PET fibers, having a linear density of about 10 dtex.

### 2. Second Layer-Coform Fibrous Structure Layer

The absorbent articles, for example absorbent articles of the present invention comprise a coform fibrous structure, for example a coform fibrous structure.

The coform fibrous structures of the present invention comprise a plurality of filaments and a plurality of fibers. The filaments and the fibers may be mixed and/or commingled together to form the coform fibrous structure. The filaments may be present in the coform fibrous structures of the present invention at a level of less than 90% and/or less than 80% and/or less than 65% and/or less than 50% and/or greater than 5% and/or greater than 10% and/or greater than 20% and/or from about 10% to about 50% and/or from about 25% to about 45%, specifically including all values within these ranges and any ranges created thereby, by weight of the coform fibrous structure on a dry basis.

The fibers may be present in the coform fibrous structures of the present invention at a level of greater than 10% and/or greater than 25% and/or greater than 50% and/or less than 100% and/or less than 95% and/or less than 90% and/or less than 85% and/or from about 30% to about 95% and/or from about 50% to about 85%, specifically including all values within these ranges and any ranges created thereby, by weight of the coform fibrous structure on a dry basis.

The filaments and fibers may be present in the coform fibrous structures of the present invention at a weight ratio of filaments to fibers of greater than 10:90 and/or greater than 20:80 and/or less than 90:10 and/or less than 80:20 and/or from about 25:75 to about 50:50 and/or from about 30:70 to about 45:55, specifically including all values within these ranges and any ranges created thereby. In one example, the filaments and fibers are present in the coform fibrous structures of the present invention at a weight ratio of filaments to fibers of greater than 0 but less than 1.

In one example, the coform fibrous structures of the present invention exhibit a basis weight of from about 30 gsm to about 200 gsm and/or from about 30 gsm to about 175 gsm and/or from about 30 gsm to about 150 gsm and/or from about 30 gsm to about 130 gsm and/or from about 30 gsm to about 120 gsm, specifically including all values within these ranges and any ranges created thereby, as measured according to the Basis Weight Test Method described herein.

In one example as shown in FIGS. 3 and 4, a coform fibrous structure, for example a coform fibrous structure, of the second layer 30, comprises a plurality of filaments 32, for example thermoplastic polymer filaments, for example polyolefin filaments, such as a polypropylene filaments and polyethylene filaments, polyester filaments, polyesteramide filaments, polycaprolactone filaments, polyhydroxyalkanoate filaments, polylactic acid filaments, biocompatible filaments, compostable filaments and mixtures thereof and a plurality of fibers 34, such as pulp fibers, for example wood pulp fibers. In one example, the thermoplastic polymer filaments comprise polyolefin filaments, such polypropylene filaments, polypropylene copolymer filaments, polyethylene filaments, polyethylene copolymer filaments, and mixtures thereof. In another example, the filaments may comprise polyester filaments.

In one example, the filaments 32 comprise a polymer comprising a polymer chain disrupter, for example a propylene/ethylene block copolymer such as Vistamaxx from ExxonMobil. The plurality of fibers 34 may be dispersed, for example randomly throughout the filaments 32. The coform fibrous structure of the second layer 30 may further comprise a scrim component (not shown) comprising a plurality of scrim filaments and may be void or substantially void of fibers. The scrim filaments may be the same and/or different for example in chemical composition as the filaments 32 and which are deposited, for example spun, onto one or more surfaces of the coform fibrous structure of the second layer 30. The scrim component, when present, may be thermally bonded to the coform fibrous structure of the second layer 30.

In one example, the coform fibrous structure of the second layer 30 exhibits a basis weight of at least 30 gsm to about 200 gsm and/or to about 175 gsm and/or to about 150 gsm and/or to about 130 gsm and/or to about 120 gsm, specifically including all values within these ranges and any ranges created thereby, as measured according to the Basis Weight Test Method described herein.

In one example, the coform fibrous structure of the second layer 30 comprises a plurality of wood pulp fibers and/or non-wood pulp fibers.

In one example, the scrim component, when present, exhibits a basis weight of 10 gsm or less and/or less than 10 gsm and/or less than 8 gsm and/or less than 6 gsm and/or greater than 5 gsm and/or less than 4 gsm and/or greater than 0 gsm and/or greater than 1 gsm, specifically including all values within these ranges and any ranges created thereby, as measured according to the Basis Weight Test Method described herein. In one example, the scrim component may be added to the coform fibrous structure opposite the side adjacent to the base nonwoven substrate.

In one example, at least one of the scrim filaments, when present, exhibits an average diameter of less than 50 and/or less than 25 and/or less than 10 and/or at least 1 and/or greater than 1 and/or greater than 3 μm, specifically including all values within these ranges and any ranges created thereby, as measured according to the Average Diameter Test Method described herein.

In one example, the average diameter of the filaments 32 of the coform fibrous structure is less than 250 μm and/or less than 200 μm and/or less than 150 μm and/or less than 100 μm and/or less than 50 μm and/or less than 30 μm and/or less than 25 μm and/or less than 10 μm and/or greater than 1 μm and/or greater than 3 μm and/or from about 4 μm to about 20 μm and/or from about 8 μm to about 20 μm and/or from about 10 μm to about 15 μm as measured according to the Average Diameter Test Method described herein. In another example, where the layered fluid acquisition/distribution system is intended to absorb and then store fluid rather than absorb fluid and drain to a fluid storage system the average diameter of the filaments 32 of the coform fibrous structure is from about 4 μm to about 10 μm as measured according to the Average Diameter Test Method described herein.

In one example, the linear density of the filaments 32 of the coform fibrous structure is from about 0.11 dtex to about 2.8 dtex and/or from about 0.11 dtex to about 1.6 dtex and/or from about 0.11 dtex to about 0.7 dtex.

In one example, the coform fibrous structures of the present invention may comprise any suitable amount of filaments and any suitable amount of fibers. For example, the coform fibrous structures may comprise from about 10% to about 70% and/or from about 20% to about 60% and/or from about 30% to about 50% by dry weight of the coform fibrous structure of filaments and from about 90% to about 30% and/or from about 80% to about 40% and/or from about 70% to about 50% by dry weight of the coform fibrous structure of fibers, such as wood pulp fibers.

In one example, the filaments and fibers of the present invention may be present in the coform fibrous structures according to the present invention at weight ratios of filaments to fibers of from at least about 1:1 and/or at least about 1:1.5 and/or at least about 1:2 and/or at least about 1:2.5 and/or at least about 1:3 and/or at least about 1:4 and/or at least about 1:5 and/or at least about 1:7 and/or at least about 1:10.

In one example, the fibers, for example pulp fibers, such as wood pulp fibers, may be present in the coform fibrous structure at a weight ratio of softwood pulp fibers to hardwood pulp fibers of from 100:0 and/or from 90:10 and/or from 86:14 and/or from 80:20 and/or from 75:25 and/or from 70:30 and/or from 60:40 and/or about 50:50 and/or to 0:100 and/or to 10:90 and/or to 14:86 and/or to 20:80 and/or to 25:75 and/or to 30:70 and/or to 40:60. In one example, the weight ratio of softwood pulp fibers to hardwood pulp fibers is from 86:14 to 70:30.

Non-limiting examples of suitable polypropylenes for making the fibrous elements, for example filaments of the present invention are commercially available from LyondellBasell and Exxon-Mobil.

Any hydrophobic or non-hydrophilic materials within the coform fibrous structure, such as the thermoplastic filaments, for example polypropylene filaments, may be surface treated and/or melt treated with a hydrophilic modifier or include a hydrophilic melt additive. Non-limiting examples of surface treating hydrophilic modifiers include surfactants, such as Triton X-100. Non-limiting examples of melt treating hydrophilic modifiers that are added to the thermoplastic polymer composition (polymer melt), such as the polypropylene melt, prior to spinning filaments, include hydrophilic modifying melt additives such as VW351 and/or S-1416 commercially available from Polyvel, Inc. and Irgasurf commercially available from Ciba. The hydrophilic modifier may be associated with the hydrophobic or non-hydrophilic material at any suitable level known in the art. In one example, the hydrophilic modifier is associated with the thermoplastic polymer composition, such as the hydrophobic and/or non-hydrophilic material within the polymer composition at a level of greater than 0% to less than about 20% and/or greater than 0% to less than about 15% and/or greater than 0.1% to less than about 10% and/or greater than 0.1% to less than about 5% and/or greater than 0.5% to less than about 3% by dry weight of the hydrophobic or non-hydrophilic material. In another example, the hydrophilic modifier may be present in the filaments at a level of from about 0.1% to about 10% and/or from about 0.5% to about 7% and/or from about 1% to about 5% by weight of the filaments.

a. Method/Process for Making a Layered Fluid Acquisition/Distribution System

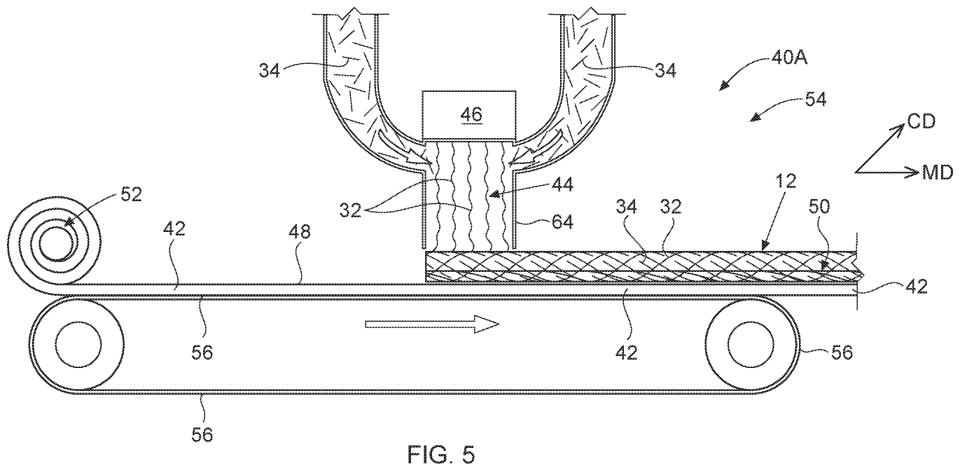

In one example, a suitable process 40A for making a layered fluid acquisition/distribution system 12 according to the present invention is shown in FIG. 5. The process 40A comprises the steps of: a) providing a base nonwoven substrate 42, which forms a first layer 28 of a layered fluid acquisition/distribution system 12 as shown in FIG. 3; b) depositing and/or collecting a mixture 44 of fibers 34 and filaments 32 (filaments 32 spun from a filament source 46, such as a die, for example a multi-row capillary die and/or a knife edge die), for example a coform mixture of fibers 34 and filaments 32, onto a surface 48 of the base nonwoven substrate 42 such that a coform fibrous structure is formed as a second layer 30 of the layered fluid acquisition/distribution system 12 as shown in FIG. 3.

In one example, the step of providing a base nonwoven substrate 42, which forms a first layer 28 as shown in FIG. 3, comprises unwinding a roll 52 of the base nonwoven substrate 42 on a layered fluid acquisition/distribution system manufacturing line 54. During the process 40A, the base nonwoven substrate 42 may be carried on a belt 56 while the mixture 44 of fibers 34 and filaments 32 wherein the filaments 32 are spun from a die and mixed with fibers 34 after which the mixture 44 of fibers 34 and filaments 32 is then laid directly on (deposited and/or collected) on a surface 48 of the base nonwoven substrate 42 such that a coform fibrous structure 50 is formed on the surface 48 of the base nonwoven substrate 42 resulting in the formation of a layered fluid acquisition/distribution system 12.

This step of depositing and/or collecting the mixture 44 on the surface 48 of the base nonwoven substrate 42 may comprise subjecting the resulting layered fluid acquisition/distribution system 12 to a consolidation step and/or a thermal bonding step whereby the layered fluid acquisition/distribution system 12 is pressed between a nip (not shown), for example a nip formed by a flat or even surface rubber roll and a flat or even surface or patterned, heated (with oil) or unheated metal roll.

In one example, the step of depositing and/or collecting the mixture 44 on the surface 48 of the base nonwoven substrate 42 may be vacuum assisted by a vacuum box (not shown) under the belt 56.

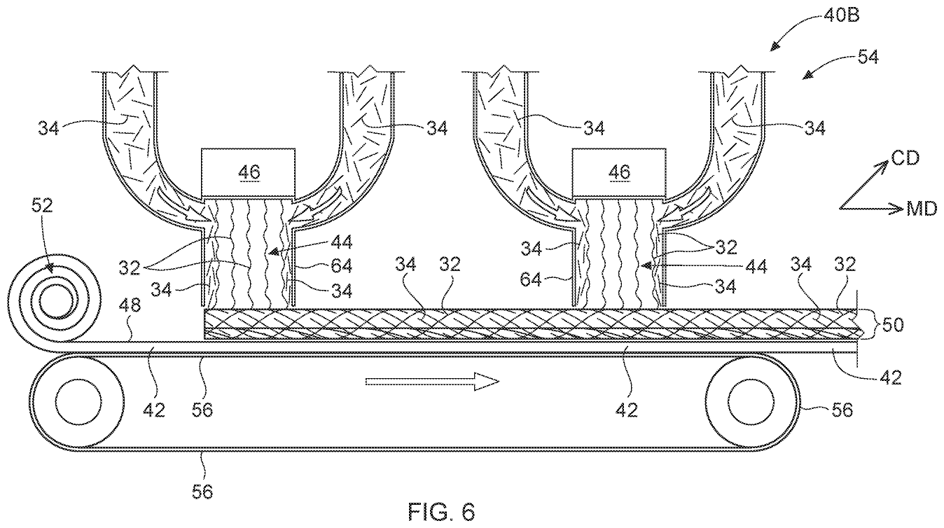

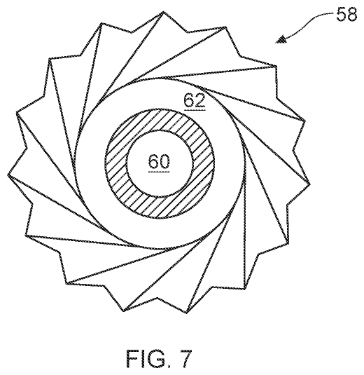

In another example, a suitable process 40B for making a layered fluid acquisition/distribution system 12 according to the present invention is shown in FIG. 6. The process 40B comprises the steps of: a) providing a base nonwoven substrate 42, which forms a first layer 28 of a layered fluid acquisition/distribution system 12 as shown in FIG. 4; b) depositing and/or collecting a first mixture 44 of fibers 34 and filaments 32 (filaments 32 spun from a filament source 46, such as a die, for example a multi-row capillary die and/or a knife edge die), for example a coform mixture of fibers 34 and filaments 32, onto a surface 48 of the base nonwoven substrate 42 such that a first layer of a layered coform fibrous structure 50 is formed; c) depositing and/or collecting a second mixture 44 of fibers 34 and filaments 32 (filaments 32 spun from a filament source 46, such as a die, for example a multi-row capillary die and/or a knife edge die), for example a coform mixture of fibers 34 and filaments 32, onto the first layer of the layered coform fibrous structure 50 that was previously deposited onto a surface 48 of the base nonwoven substrate 42 such that a second layer of a layered coform fibrous structure 50 is formed, which results in a layered fluid acquisition/distribution system 12 comprising a first layer 28 comprising the base nonwoven substrate 42 and a second layer 30 comprising a layered coform fibrous structure 50 as shown in FIG. 4. The first layer of the layered coform fibrous structure 50 may exhibit different properties than the second layer of the layered coform fibrous structure 50. In one example, the filaments 32 of the first layer of the layered coform fibrous structure 50 exhibit a greater average diameter than the filaments 32 of the second layer of the layered coform fibrous structure 50. In one example, the second layer of the layered coform fibrous structure 50 exhibits a greater capillarity than the first layer of the layered coform fibrous structure 50. In one example, the second layer of the layered coform fibrous structure 50 exhibits a higher density than the first layer of the layered coform fibrous structure 50.

In one example, the step of providing a base nonwoven substrate 42, which forms a first layer 28 as shown in FIG. 4, comprises unwinding a roll 52 of the base nonwoven substrate 42 on a layered fluid acquisition/distribution system manufacturing line 54. During the process 40B, the base nonwoven substrate 42 may be carried on a belt 56 while the mixture 44 of fibers 34 and filaments 32 wherein the filaments 32 are spun from a die and mixed with fibers 34 after which the first mixture 44 of fibers 34 and filaments 32 is then laid directly on (deposited and/or collected) on a surface 48 of the base nonwoven substrate 42 such that a first layer of a layered coform fibrous structure 50 is formed on the surface 48 of the base nonwoven substrate 42 and then a second layer of the layered coform fibrous structure 50, similar to the first layer of the layered coform fibrous structure 50, is then spun, mixed and then laid directly on (deposited and/or collected) on the previously formed first layer of the layered coform fibrous structure 50 resulting in the formation of a layered fluid acquisition/distribution system 12.

This step of depositing and/or collecting the mixture 44 on the surface 48 of the base nonwoven substrate 42 may comprise subjecting the resulting layered fluid acquisition/distribution system 12 to a consolidation step and/or a thermal bonding step whereby the layered fluid acquisition/distribution system 12 is pressed between a nip (not shown), for example a nip formed by a flat or even surface rubber roll and a flat or even surface or patterned, heated (with oil) or unheated metal roll.

In one example, the step of depositing and/or collecting the mixture 44 on the surface 48 of the base nonwoven substrate 42 may be vacuum assisted by a vacuum box (not shown) under the belt 56.

In one example, the filament source 46 of the process 40A and 40B may be a meltblown die, for example a multi-row capillary die, a knife-edge die, and combinations thereof. In one example, the filament source 46 is a meltblown die. In one example, the meltblown die is a multi-row capillary die 58 comprising a plurality of filament-forming holes 60, one of which is shown in FIG. 7, which is positioned coaxially within a fluid-releasing hole 62 that provides attenuation air to the thermoplastic polymer exiting the filament-forming holes 60 such that a plurality of filaments 32 are formed by the multi-row capillary die 58. The fluid-releasing hole 62 may be concentrically or substantially concentrically positioned around the filament-forming hole 60. In one example, the fluid, for example attenuation air, exits one of more, for example each fluid-releasing hole 62 parallel or substantially parallel to the filament 32 exiting the one or more filament-forming holes 60.

In one example of the present invention, the process for making a layered fluid acquisition/distribution system comprises the steps of:
a. providing a plurality of filaments;
b. providing a plurality of fibers; and
c. mixing and/or commingling the plurality of filaments with the plurality of fibers;
d. depositing and/or collecting the mixed (commingled) plurality of filaments and plurality of fibers on a surface of a base nonwoven substrate, wherein the base nonwoven substrate may be carried on a belt, such that a layered fluid acquisition/distribution system is formed.

In one example, the process comprises the steps of: a) providing one or more streams of filaments comprising a plurality of filaments, one or more streams of fibers comprising a plurality of fibers all streams being separate from one another and/or neat streams (for example less than 10% and/or less than 5% and/or less than 3% and/or about 0% and/or 0% by weight of material different from their respective materials); b) mixing and/or commingling the plurality of fibers with the plurality of filaments; and c) depositing and/or collecting the mixed (commingled) filaments and fibers on a surface of a base nonwoven substrate such that a layered fluid acquisition/distribution system 12 is formed.

As shown in FIGS. 5 and 6, the step of mixing and/or commingling of the filaments and fibers may occur within an enclosure 64 (housing), for example a forming box, such as a coforming box.

In one example, the step of mixing and/or commingling the plurality of filaments with the plurality of fibers comprises introducing the plurality of fibers into a stream of the plurality of filaments at an angle of from about 10° to about 170° and/or from about 20° to about 150° and/or from about 30° to about 130° and/or from about 30° to about 120° and/or from about 45° to about 100° and/or from about 60° to about 90° relative to the stream of the plurality of filaments.

In one example, the plurality of fibers are non-uniformly distributed within the layered fluid acquisition/distribution system such that there are a greater number of fibers concentrated near one side of the layered fluid acquisition/distribution system and/or one side of the coform fibrous structure of the layered fluid acquisition/distribution system than the number of fibers concentrated on an opposite side and/or another z-direction region of the layered fluid acquisition/distribution system and/or coform fibrous structure thereof.

In one example, a process for making a layered fluid acquisition/distribution system according to the present invention comprises the steps of: 1) collecting a mixture of filaments and fibers, for example pulp fibers, onto a base nonwoven substrate, for example carried on a belt, for example a through-air-drying fabric or other fabric or a patterned molding member of the present invention.

In another example of the present invention, the process for making a layered fluid acquisition/distribution system comprises the steps of:
a. providing a base nonwoven substrate;
b. providing a pre-formed coform fibrous structure comprising a plurality of filaments and a plurality of fibers commingled together; and
c. associating the pre-formed coform fibrous structure with a surface of the base nonwoven substrate such that a layered fluid acquisition/distribution system is formed.

### Non-Limiting Example for Making a Layered Fluid Acquisition/Distribution System

A base nonwoven substrate, for example a carded base nonwoven substrate, is unwound from a roll onto a belt traveling at 556 ft/min of a layered fluid acquisition/distribution system manufacturing line.

A coform fibrous structure is deposited and collected and formed directly on a surface of the base nonwoven substrate while the base nonwoven substrate is traveling on the belt of the layered fluid acquisition/distribution system manufacturing line. The coform fibrous structure is made as follows: A plurality of fibers, in this case pulp fibers, namely, 490 grams per minute of Resolute CoosAbsorbST semi-treated SSK, are fed into a hammer mill and individualized into fibers, for example cellulose pulp fibers, which are pneumatically conveyed, for example by an eductor, example of which is described in U.S. Patent Publication No. US 2016/0354736A1, into a forming box, such as a coforming box, example of which is described in U.S. Patent Publication No. US 2016/0355950A1 filed Dec. 16, 2015, which is incorporated herein by reference. In the forming box, the fibers are mixed and commingled with a plurality of meltblown filaments. The meltblown filaments are comprised of a blend of 45.4% LyondellBasell MF650x (polypropylene), 26.5% LyondellBasell MF650w (polypropylene), 16.1% Total 3866 (polypropylene), 5% Polyvel VW351 (hydrophilic modifier), 2% Ampacet 412951 (opacifier), and 5% Vistamaxx 7050FL (polymer chain disrupter). The meltblown filaments are spun from a die, for example a multi-row capillary Biax-Fiberfilm die, at a ghm of 0.206 and a total mass flow of 126.7 g/min. The meltblown filaments are attenuated with 15.65 kg/min of about 204° C. (400° F.) air. The mixed (commingled) fibers, for example cellulose pulp fibers, and meltblown filaments are then laid on top of the surface of the base nonwoven substrate to form the layered fluid acquisition/distribution system comprising a first layer comprising the base nonwoven substrate and a second layer comprising a coform fibrous structure. In one example, the base nonwoven substrate of the present invention may exhibit a basis weight of from about 10 gsm to about 60 gsm. In one example, the coform fibrous structure of the present invention may exhibit a basis weight of from about 30 to about 200 gsm.

a. Filaments of Coform Fibrous Structure Layer

The filaments may comprise a polymer, for example a thermoplastic polymer, such as a thermoplastic polymer is selected from the group consisting of: polyolefins, polyesters, polyesteramides, polycaprolactones, polyhydroxyalkanoates, polylactic acids, and mixtures thereof. In one example, the thermoplastic polymer is a polyolefin, such as a polyolefin selected from the group consisting of: polypropylene, polypropylene copolymers, polyethylene, polyethylene copolymers, and mixtures thereof.

In one example, the thermoplastic polymer is a biodegradable thermoplastic polymer.

In one example, the thermoplastic polymer is a compostable thermoplastic polymer.

Non-limiting examples of suitable polypropylenes for making the filaments, for example filaments of the present invention are commercially available from LyondellBasell and Exxon-Mobil.

Any hydrophobic or non-hydrophilic materials within the coform fibrous structure, such as the thermoplastic filaments, for example the polypropylene filaments, may be surface treated and/or melt treated with a hydrophilic modifier or include a hydrophilic melt additive. Non-limiting examples of surface treating hydrophilic modifiers include surfactants, such as Triton X-100. Non-limiting examples of melt treating hydrophilic modifiers that are added to the polymer composition (polymer melt), such as the polypropylene melt, prior to spinning filaments, include hydrophilic modifying melt additives such as VW351 and/or S-1416 commercially available from Polyvel, Inc. and Irgasurf commercially available from Ciba. The hydrophilic modifier may be associated with the hydrophobic or non-hydrophilic material at any suitable level known in the art. In one example, the hydrophilic modifier is associated with the polymer composition, such as the hydrophobic and/or non-hydrophilic material within the polymer composition at a level of greater than 0% to less than about 20% and/or greater than 0% to less than about 15% and/or greater than 0.1% to less than about 10% and/or greater than 0.1% to less than about 5% and/or greater than 0.5% to less than about 3% by dry weight of the hydrophobic or non-hydrophilic material. In another example, the hydrophilic modifier may be present in the filaments at a level of from about 0.1% to about 10% and/or from about 0.5% to about 7% and/or from about 1% to about 5% by weight of the filaments.

b. Fibers of Coform Fibrous Structure Layer

In one example, the fibers of the present invention, for example pulp fibers, for example wood pulp fibers, may be selected from the group consisting of softwood kraft pulp fibers, hardwood pulp fibers, and mixtures thereof. Non-limiting examples of hardwood pulp fibers include fibers derived from a fiber source selected from the group consisting of: Acacia, Eucalyptus, Maple, Oak, Aspen, Birch, Cottonwood, Alder, Ash, Cherry, Elm, Hickory, Poplar, Gum, Walnut, Locust, Sycamore, Beech, Catalpa, Sassafras, Gmelina, Albizia, Anthocephalus, and Magnolia. Non-limiting examples of softwood pulp fibers include fibers derived from a fiber source selected from the group consisting of: Pine, Spruce, Fir, Tamarack, Hemlock, Cypress, and Cedar. In one example, the hardwood pulp fibers comprise tropical hardwood pulp fibers. Non-limiting examples of suitable tropical hardwood pulp fibers include Eucalyptus pulp fibers, Acacia pulp fibers, and mixtures thereof.

In one example, the wood pulp fibers comprise softwood pulp fibers derived from the kraft process and originating from southern climates, such as Southern Softwood Kraft (SSK) pulp fibers. In another example, the wood pulp fibers comprise softwood pulp fibers derived from the kraft process and originating from northern climates, such as Northern Softwood Kraft (NSK) pulp fibers.

The wood pulp fibers, when present in the process and/or structure of the present invention may be present at a weight ratio of softwood pulp fibers to hardwood pulp fibers of from 100:0 and/or from 90:10 and/or from 86:14 and/or from 80:20 and/or from 75:25 and/or from 70:30 and/or from 60:40 and/or about 50:50 and/or to 0:100 and/or to 10:90 and/or to 14:86 and/or to 20:80 and/or to 25:75 and/or to 30:70 and/or to 40:60. In one example, the weight ratio of softwood pulp fibers to hardwood pulp fibers is from 86:14 to 70:30.

In one example, the fibers of the coform fibrous structure comprise one or more non-wood pulp fibers, for example one or more trichomes. Non-limiting examples of suitable sources for obtaining trichomes, especially trichome fibers, are plants in the labiatae (Lamiaceae) family commonly referred to as the mint family. Examples of suitable species in the labiatae family include Stachys byzantina, also known as Stachys lanata commonly referred to as lamb's ear, woolly betony, or woundwort. The term Stachys byzantina as used herein also includes cultivars Stachys byzantina ‘Primrose Heron’, Stachys byzantina ‘Helene von Stein’ (sometimes referred to as Stachys byzantina ‘Big Ears’), Stachys byzantina ‘Cotton Boll’, Stachys byzantina ‘Variegated’ (sometimes referred to as Stachys byzantina ‘Striped Phantom’), and Stachys byzantina ‘Silver Carpet’.

In another example, the fibers of the coform fibrous structure may comprise one or more super absorbent polymer fibers.

The fibers may comprise pulp fibers. “Pulp fibers” as used herein means fibers that have been derived from plant sources. In one example within contemplation of the present disclosure, “pulp fiber” refers to papermaking fibers. In one example within contemplation of the present disclosure, a fiber may be a naturally occurring fiber, which means it is obtained from a plant source such as one or more species of trees. Such fibers are typically used in papermaking and are oftentimes referred to as papermaking fibers. Papermaking fibers useful within contemplation of the present disclosure include cellulose fibers commonly known as wood pulp fibers. Applicable wood pulps include chemical pulps, such as Kraft, sulfite, and sulfate pulps, as well as mechanical pulps including, for example, groundwood, thermomechanical pulp and chemically modified thermomechanical pulp. Chemical pulps, however, may be preferred since they impart a superior tactile sense of softness to fibrous structures made therefrom. Pulps derived from both deciduous trees (hereinafter, also referred to as “hardwood”) and coniferous trees (hereinafter, also referred to as “softwood”) may be utilized. Hardwood pulp fibers may be selected from the group consisting of eucalyptus fibers, acacia fibers, aspen fibers, birch fibers, maple fibers and mixtures thereof. Softwood fibers may be selected from the group consisting of cedar fibers, fir fibers, pine fibers, spruce fibers and mixtures thereof. Hardwood and softwood fibers can be blended in a single stratum or zone, or alternatively, can be deposited in layers to provide a stratified web. Also useful within contemplation of the present disclosure are fibers derived from recycled paper, which may contain any or all of the above categories of fibers as well as other non-fibrous polymers such as fillers, softening agents, wet and dry strength agents, and adhesives used to facilitate the original papermaking.

In addition to the various wood pulp fibers, other cellulose or cellulose-derived fibers such as cotton, cotton linters, rayon, lyocell, viscose, rice straw, wheat straw, bamboo, and bagasse fibers are within contemplation of the present disclosure. Other sources of cellulose in the form of fibers or material that may be processed and spun into fibers include grain/cercal stalks (e.g., wheat, rye, corn, sorghum, Hesperaloe funifera, etc.), kapok, milkweed, coconut husk, kenaf, jute, flax, ramie, hemp, abaca, sisal, grasses (e.g., esparto, lemon, sabia, switchgrass, etc.), and canes (e.g., bamboo, bagasse, etc.).

In one example, polymeric fibers, for example staple fibers, such as thermoplastic polymer fibers, such as polyester fibers, nylon fibers, polyolefin fibers such as polypropylene fibers, polyethylene fibers, propylene/ethylene copolymer fibers, and biodegradable or compostable thermoplastic fibers such as polylactic acid fibers, polyhydroxyalkanoate fibers, polyesteramide fibers, and polycaprolactone fibers. The polymeric fibers may be monocomponent or multicomponent, such as bicomponent filaments.

### Topsheet

The topsheet may be formed of any known or otherwise effective web material, such as one which is compliant, soft feeling, and non-irritating to a wearer's skin. Suitable topsheet materials include any liquid pervious material that when placed in contact with the body of the wearer will permit bodily discharges to pass from the wearer-facing surface to the outward-facing surface of the topsheet. The topsheet, in addition to being configured to allow movement of fluid through it, may also provide for the transfer or migration of lotion composition onto a wearer's skin. A suitable topsheet can be made of various materials such as woven and nonwoven materials; apertured film materials including apertured formed thermoplastic films, apertured plastic films, and fiber-entangled apertured films; hydro-formed thermoplastic films; porous foams; reticulated foams; reticulated thermoplastic films; thermoplastic scrims; or combinations thereof.

Apertured film materials suitable for use to form a topsheet include those apertured plastic films that are non-absorbent and pervious to body exudates and provide for minimal or no flow back of fluids through the topsheet. Nonlimiting examples of other suitable formed films, including apertured and non-apertured formed films, are more fully described in U.S. Pat. Nos. 3,929,135; 4,324,246; 4,342,314; 4,463,045; 5,006,394; 4,609,518; and 4,629,643. Commercially available formed filmed topsheets include those topsheet materials marketed by the Procter & Gamble Company (Cincinnati, Ohio) under the trade name/trademark DRI-WEAVE.

Other nonlimiting examples suitable for use to form a topsheet may include woven and nonwoven materials, including fibrous materials made from natural fibers, modified natural fibers, synthetic fibers, or combinations thereof. These fibrous materials can be either hydrophilic or hydrophobic, but it is preferable that the topsheet be at least partially hydrophobic or rendered at least partially hydrophobic. As an option, portions of the topsheet can be rendered hydrophilic, using any known method for making topsheets containing hydrophilic constituents. One such method includes treating an apertured film component of a nonwoven/apertured thermoplastic formed film topsheet with a surfactant as described in U.S. Pat. No. 4,950,264. Other suitable methods and processes for treating the topsheet with a surfactant are described in U.S. Pat. Nos. 4,988,344 and 4,988,345. The topsheet may include hydrophilic fibers, hydrophobic fibers, or combinations thereof.

A particularly suitable topsheet may include staple-length polypropylene fibers having a denier of about 1.5, such as Hercules type 151 polypropylene marketed by Hercules, Inc. of Wilmington, Delaware. As used herein, the term “staple-length fibers” refers to those fibers having a length of at least about 15.9 mm (0.62 inches).

When the topsheet includes or is formed of a nonwoven fibrous material in the form of a nonwoven web, the nonwoven web may be produced by any known procedure for making nonwoven webs, nonlimiting examples of which include spunbond processes, carding, wet-laying, air-laying, meltblowing, needle-punching, mechanical entangling, thermo-mechanical entangling, and spunlacing or hydroentangling. A specific example of a suitable meltblowing process is disclosed in U.S. Pat. No. 3,978,185. The nonwoven may be compression resistant as described in U.S. Pat. No. 7,785,690. The nonwoven web may be manufactured to have loops as described in U.S. Pat. No. 7,838,099.

Other suitable nonwoven materials include low basis weight nonwovens, that is, nonwovens having a basis weight of from about 18 gsm to about 25 gsm. An example of such a nonwoven material is commercially available under the trade name P-8 from Veratec, Inc., a division of the International Paper Company in Walpole, Massachusetts. Other nonwovens are described in U.S. Pat. Nos. 5,792,404 and 5,665,452.

The topsheet may have tufts as described in U.S. Pat. Nos. 8,728,049; 7,553,532; 7,172,801; or 8,440,286. The topsheet may have an inverse textured web as described in U.S. Pat. No. 7,648,752. Tufts are also described in U.S. Pat. No. 7,410,683.

The topsheet may have a pattern of discrete hair-like fibrils as described in U.S. Pat. No. 7,655,176 or U.S. Pat. No. 7,402,723.

The topsheet may include one or more structurally modified zones as described in U.S. Pat. No. 8,614,365. The topsheet may include one or more out-of-plane deformations as described in U.S. Pat. No. 8,704,036. The topsheet may have a masking composition as described in U.S. Pat. No. 6,025,535.

Another suitable topsheet material or a topsheet material combined with an acquisition layer material may be formed from a three-dimensional substrate as described in U.S. provisional application Ser. No. 62/306,676. This three-dimensional substrate has a first surface, a second surface, and land areas, and also includes three-dimensional protrusions extending outward from the second surface of the three-dimensional substrate, wherein the three-dimensional protrusions are surrounded by the land areas. The substrate is a laminate comprising at least two layers in a face to face relationship, the second layer is a tissue layer facing outward from the second surface of the three-dimensional substrate, and the tissue layer comprises at least 80% pulp fibers by weight of the tissue layer.

The topsheet may include one or more layers, for example, a spunbond-meltblown-spunbond material. The topsheet may be apertured, may have any suitable three-dimensional features, and/or may have a plurality of embossments (e.g., a bond pattern). The topsheet may be apertured by overbonding a material and then rupturing the overbonds through ring rolling, such as disclosed in U.S. Pat. No. 5,628,097.

Added lateral extensibility in the absorbent article (i.e., in the topsheet and/or the backsheet) may be provided in a variety of ways. For example, either the topsheet or backsheet may be pleated by any known methods. Alternatively, all or a portion of the article (i.e., the topsheet and/or backsheet) may be made of a formed web material or a formed laminate of web materials like those described in U.S. Pat. No. 5,518,801. Such a formed web material includes distinct laterally extending regions in which the original material has been altered by embossing or another method of deformation to create a pattern of generally longitudinally oriented alternating ridges and valleys. The formed web material also includes laterally extending unaltered regions located between the laterally extending altered regions.

### Backsheet

The backsheet may be impervious, or substantially impervious, to liquids (e.g., urine) and may be manufactured from a thin plastic film, although other flexible liquid impervious materials may also be used. As used herein, the term “flexible” refers to materials which are compliant and will readily conform to the general shape and contours of the human body. The backsheet 30 may prevent, or at least inhibit, the exudates absorbed and contained in the absorbent core structure 40 from wetting articles of clothing which contact the incontinence pad 10 such as undergarments. However, in some instances, the backsheet may permit vapors to escape from the absorbent core structure (i.e., is breathable) while in other instances the backsheet may not permit vapors to escape (i.e., non-breathable). Thus, the backsheet may comprise a polymeric film such as thermoplastic films of polyethylene or polypropylene. A suitable material for the backsheet is a thermoplastic film having a thickness of from about 0.012 mm (0.5 mil) to about 0.051 mm (2.0 mils), for example. Any suitable backsheet known in the art may be utilized with the absorbent article of the present invention.

The backsheet acts as a barrier to any absorbed bodily fluids that may pass through the absorbent core structure to the outward-facing surface thereof with a resulting reduction in risk of staining undergarments or other clothing. A preferred material is a soft, smooth, compliant, liquid and vapor pervious material that provides for softness and conformability for comfort, and is low noise producing so that movement does not cause unwanted sound.

The backsheet may comprise a wet laid fibrous assembly having a temporary wet strength resin incorporated therein as described in U.S. Pat. No. 5,885,265. The backsheet may further be coated with a water resistant resinous material that causes the backsheet to become impervious to bodily fluids without impairing the spreading of adhesive materials thereon.

Another suitable backsheet material is a polyethylene film having a thickness of from about 0.012 mm (0.5 mil) to about 0.051 mm (2.0 mils). The backsheet may be embossed and/or matte finished to provide a more cloth-like appearance. Further, the backsheet may permit vapors to escape from the absorbent structure 42 (i.e., the backsheet is breathable) while still preventing body fluids from passing through the backsheet. A preferred microporous polyethylene film which is available from Tredegar Corporation, Virginia, USA, under Code No. XBF-1 12W.

For a stretchable but non-elastic backsheet, one material can be used is a hydrophobic, stretchable, spun laced, non-woven material having a basis weight of from about 30 to 40 gsm, formed of polyethylene terephthalate or polypropylene fibers. This material is breathable, i.e., permeable to water vapor and other gases.

For an elastic backsheet, one material which can be used is an elastic film sold under the trademark EXX500 by Exxon Corporation. The material of this film is formed from an elastomeric base composition consisting of a styrene block copolymer. However, this material is not breathable. Another material which can be used for an elastic backsheet is a plastic film that has been subjected to a process that provides it with elastic-like properties without attaching elastic strands to the film, and may for example comprise a formed film made in accordance with U.S. Pat. Nos. 4,342,314 and 4,463,045.

Suitable breathable backsheets for use herein include all breathable backsheets known in the art. In principle there are two types of breathable backsheets, single layer breathable backsheets which are breathable and impervious to liquids and backsheets having at least two layers, which in combination provide both breathability and liquid imperviousness. Suitable single layer breathable backsheets for use herein include those described for example in GB A 2184 389; GB A 2184 390; GB A 2184 391; U.S. Pat. Nos. 4,591,523; 3,989,867; 3,156,242; and WO 97/24097.

The backsheet may have two layers: a first layer comprising a gas permeable aperture formed film layer and a second layer comprising a breathable microporous film layer as described in U.S. Pat. No. 6,462,251. Suitable dual or multi-layer breathable backsheets for use herein include those exemplified in U.S. Pat. Nos. 3,881,489; 4,341,216; 4,713,068; 4,818,600; EP 203 821; EP 710 471; EP 710 472; and EP 793 952.

The backsheet may be a relatively hydrophobic, 18 gsm spunbond nonwoven web of 2 denier polypropylene fibers. The backsheet may also be a laminate as is known in the art.

The backsheet may be vapor permeable as described in U.S. Pat. No. 6,623,464, or U.S. Pat. No. 6,664,439. The backsheet can be formed from any vapor permeable material known in the art. The backsheet may be a microporous film, an apertured formed film, or other polymer film that is vapor permeable, or rendered to be vapor permeable, as is known in the art.

The backsheet may be a nonwoven web having a basis weight between about 20 gsm and about 50 gsm. In one embodiment, the backsheet is a relatively hydrophobic, 23 gsm spunbond nonwoven web of 4 denier polypropylene fibers available from Fiberweb Neuberger, under the designation F102301001. The backsheet may be coated with a non-soluble, liquid swellable material as described in U.S. Pat. No. 6,436,508.

The backsheet has an outward-facing side and an opposite wearer-facing side. The outward-facing side of the backsheet comprises a non-adhesive area and an adhesive area. The adhesive area may be provided by any conventional means. Pressure sensitive adhesives have been commonly found to work well for this purpose.

### Fluid Storage System—Absorbent Core

Any suitable absorbent core known in the art may be utilized. The absorbent core may be any absorbent member which is generally compressible, conformable, non-irritating to the wearer's skin, and capable of absorbing and retaining liquids such as urine, menses, and/or other body exudates. The absorbent core may be manufactured from a wide variety of liquid-absorbent materials commonly used in disposable absorbent articles such as comminuted wood pulp which is generally referred to as airfelt. The absorbent core may comprise superabsorbent polymers (SAP) and less than 15%, less than 10%, less than 5%, less than 3%, or less than 1% of airfelt, or be completely free of airfelt. Examples of other suitable absorbent materials comprise creped cellulose wadding, meltblown polymers including coform, chemically stiffened, modified or cross-linked cellulosic fibers, tissue including tissue wraps and tissue laminates, absorbent foams, absorbent sponges, superabsorbent polymers, absorbent gelling materials, or any equivalent material or combinations of materials.

The configuration and construction of the absorbent core may vary (e.g., the absorbent core may have varying caliper zones, a hydrophilic gradient, a superabsorbent gradient, or lower average density and lower average basis weight acquisition zones; or may comprise one or more layers or structures). In one example, the absorbent core may comprise one or more channels, such as two, three, four, five, or six channels.

The absorbent core of the present invention may comprise one or more adhesives, for example, to help immobilize the SAP or other absorbent materials within a core wrap and/or to ensure integrity of the core wrap, in particular when the core wrap is made of two or more substrates. The core wrap may extend to a larger area than required for containing the absorbent material(s) within.

Absorbent cores comprising relatively high amounts of SAP with various absorbent core designs are disclosed in U.S. Pat. No. 5,599,335 to Goldman et al., EP 1,447,066 to Busam et al., WO 95/11652 to Tanzer et al., U.S. Pat. Publ. No. 2008/0312622 A1 to Hundorf et al., and WO 2012/052172 to Van Malderen.

Other forms and more details regarding channels and pockets that are free of, or substantially free of absorbent materials, such as SAP, within absorbent cores are discussed in greater detail in U.S. Patent Application Publication Nos. 2014/0163500, 2014/0163506, and 2014/0163511, all published on Jun. 12, 2014.

Other suitable materials for use in absorbent cores comprise open celled foams or pieces thereof. The use of foams in absorbent cores is described in additional detail in U.S. Pat. Nos. 6,410,820; 6,107,356; 6,204,298; 6,207,724; 6,444,716; 8,211,078, and 8,702,668.

In one example, the absorbent core may comprise a heterogeneous mass layer or may utilize methods or parameters such as those described in U.S. patent application Ser. No. 14/715,984, filed May 19, 2015; U.S. patent application Ser. No. 14/750,399, Jun. 25, 2015; U.S. patent application Ser. No. 14/751,969 filed Jun. 26, 2015; U.S. patent application Ser. No. 15/078,132 filed Mar. 23, 2016; U.S. patent application Ser. No. 14/750,596 filed Jun. 25, 2015; U.S. patent application Ser. No. 15/084,902 filed Mar. 30, 2016; U.S. patent application Ser. No. 15/343,989 filed Nov. 4, 2016; U.S. patent application Ser. No. 15/344,273 filed Nov. 4, 2016; U.S. patent application Ser. No. 15/344,294 filed Nov. 4, 2016; U.S. patent application Ser. No. 14/704,110 filed May 5, 2015; U.S. patent application Ser. No. 15/194,894 filed Jun. 28, 2016; U.S. patent application Ser. No. 15/344,050 filed Nov. 4, 2016; U.S. patent application Ser. No. 15/344,117 filed Nov. 4, 2016; U.S. patent application Ser. No. 15/344,177 filed Nov. 4, 2016; U.S. patent application Ser. No. 15/344,198 filed Nov. 4, 2016; U.S. patent application Ser. No. 15/344,221 filed Nov. 4, 2016; U.S. patent application Ser. No. 15/344,239 filed Nov. 4, 2016; U.S. patent application Ser. No. 15/344,255 filed Nov. 4, 2016; U.S. patent application Ser. No. 15/464,733 filed Nov. 4, 2016; U.S. Provisional Patent Application No. 62/437,208 filed Dec. 21, 2016; U.S. Provisional Patent Application No. 62/437,225 filed Dec. 21, 2016; U.S. Provisional Patent Application No. 62/437,241 filed Dec. 21, 2016; or U.S. Provisional Patent Application No. 62/437,259 filed Dec. 21, 2016. The heterogeneous mass layer has a depth, a width, and a height.

In one example, a combination of absorbent core materials may be utilized. For example, forms are contemplated where a first layer of an absorbent core comprises a foam material or pieces thereof as described previously and a second layer of an absorbent core comprises an airlaid material. Such combinations are described in U.S. Patent Publication No. 2014/0336606 and U.S. Pat. No. 9,649,228.

In one example, traditional absorbent core materials include thick (or densified cellulose fluff) or absorbent airlaid materials mostly composed of short (2.5 mm) cellulose fibers

In one example, the absorbent core comprises a coform absorbent core, for example an absorbent core formed by commingling a plurality of filaments and superabsorbent polymer (AGM) particles and/or superabsorbent polymer (AGM) fibers having a total basis weight of greater than 100 and/or greater than 125 and/or greater than 150 and/or greater than 175 and/or greater than 200 and/or greater than 220 gsm. In one example, the coform absorbent core comprises a homogenously blend of cellulose fibers, polypropylene filaments, and superabsorbent polymer (AGM). These materials were homogenously blended.

In one example, the absorbent core comprises an airlaid absorbent core comprising a blend of airlaid materials, for example polyethylene/polyethylene terephthalate fibers, cellulose fibers, and latex binder material.

In one example, the absorbent core comprise a unitary airlaid absorbent core comprising a blend of cellulose fibers, superabsorbent polymer, which may comprise superabsorbent polymer fibers, and bondable fibers.

In one example, the absorbent core comprises a carded spunlace material comprising viscose rayon, PET/CoPET bicomponent fibers and polyethyleneterephthalate monocomponent fibers.

In one example, the absorbent core comprises a coform absorbent core comprising polypropylene and/or polyethylene and/or biodegradable polymer and/or compostable polymer filaments and superabsorbent polymer particles as described in US Patent Application Publication Nos. 2022/0133555 A1 and 2022/0133548 A1, which are incorporated herein by reference.

### Non-Limiting Examples

Materials used in the non-limiting examples include the following:

Base nonwoven substrate may be any one or more of the following: carded base nonwoven substrates commercially available from Xiamen Yanjan New Material Co., China, for example under tradenames such as Aura 20, ATB 287G-30, ATB 287G-40, Aura 66; hydroentangled spunlace base nonwoven substrates commercially available from Sandler GmbH, Germany, for example under the trademark Sawasoft®; spunbond base nonwoven substrates commercially available from PFNonwovens Czech S.R.O., Czech Republic.

Coform fibrous structure filaments may be any one or more of the following: Vistamaxx™ and polypropylene, for example under the tradename MFR, commercially available from ExxonMobile USA, and polypropylene commercially available from LyondellBasell, for example under the tradenames 650X and 650W.

The coform fibrous structure filaments may comprise a wetting agent, for example Polyvel VW351 commercially available from Polyvel.

Coform fibrous structure fibers may be any one or more of the following: GP treated southern softwood kraft pulp fibers commercially available from Georgia-Pacific, Eucafluff (eucalyptus pulp fibers) commercially available from Suzano Papel e Celulose, Brazil, and NB416 southern softwood kraft pulp fibers commercially available from International Paper.

### Non-Limiting Combinations of the Present Invention

A. A layered fluid acquisition/distribution system comprising:
   a. a base nonwoven substrate, such as a nonwoven substrate that exhibits a basis weight of from 10 gsm to 60 gsm and/or 10 gsm to 50 gsm and/or 20 gsm to 50 gsm and/or 20 gsm to 40 gsm; and
   b. a coform fibrous structure comprising a plurality of filaments and a plurality of fibers,
B. The layered fluid acquisition/distribution system according to Paragraph A above wherein the base nonwoven substrate comprises a plurality of fibrous elements, for example synthetic fibers such as regenerated cellulose fibers, which may be selected from the group consisting of: rayon fibers, viscose fibers, lyocell fibers and mixtures thereof.
C. The layered fluid acquisition/distribution system according to Paragraph B above wherein the plurality of fibrous elements of the base nonwoven substrate exhibit an average diameter of from 10 μm to 50 μm and/or from 15 μm to 30 μm as measured according to the Average Diameter Test Method.
D. The layered fluid acquisition/distribution system according to any of the preceding Paragraphs A-C above wherein the base nonwoven substrate exhibits a caliper of from 0.2 mm to 1 mm and/or 0.2 mm to 0.75 mm and/or from 0.3 mm to 0.75 mm as measured according to the Caliper Test Method.
E. The layered fluid acquisition/distribution system according to any of the preceding Paragraphs A-D above wherein the base nonwoven substrate exhibits an air permeability of between about 150 m³/m²/min and about 500 m³/m²/min as measured according to the Air Permeability Test Method.
F. The layered fluid acquisition/distribution system according to any of the preceding Paragraphs A-E above wherein the base nonwoven substrate comprises a carded base nonwoven substrate.
G. The layered fluid acquisition/distribution system according to any of the preceding Paragraphs A-F above wherein the base nonwoven substrate comprises a spunlace base nonwoven substrate.
H. The layered fluid acquisition/distribution system according to any of the preceding Paragraphs A-G above wherein the base nonwoven substrate comprises a hydroentangled base nonwoven substrate.
I. The layered fluid acquisition/distribution system according to any of the preceding Paragraphs A-H above wherein the base nonwoven substrate comprises a spunbond base nonwoven substrate.
J. The layered fluid acquisition/distribution system according to any of the preceding Paragraphs A-I above wherein the plurality of filaments and the plurality of fibers of the coform fibrous structure are commingled together.
K. The layered fluid acquisition/distribution system according to any of the preceding Paragraphs A-J above wherein the plurality of filaments of the coform fibrous structure comprises a plurality of water-insoluble filaments.
L. The layered fluid acquisition/distribution system according to any of the preceding Paragraphs A-K above wherein the plurality of filaments of the coform fibrous structure comprises a plurality of thermoplastic filaments, such as thermoplastic filaments comprising a polyester and/or wherein the thermoplastic filaments comprise a polyolefin, such as a polyolefin selected from the group consisting of: polypropylene, polyethylene, copolymers thereof, and mixtures thereof and/or wherein the thermoplastic filaments comprise a polyester and a polyolefin and/or wherein the thermoplastic filaments comprise biodegradable thermoplastic filaments and/or compostable thermoplastic filaments and/or wherein the thermoplastic filaments are selected from the group consisting of: polylactic acid filaments, polyhydroxyalkanoate filaments, polycaprolactone filaments and mixtures thereof.
M. The layered fluid acquisition/distribution system according to any of the preceding Paragraphs A-L above wherein the plurality of fibers of the coform fibrous structure comprises a plurality of pulp fibers, such as wood pulp fibers for example wood pulp fibers selected from the group consisting of: hardwood pulp fibers, softwood pulp fibers, and mixtures thereof and/or non-wood pulp fibers, such as cotton fibers, trichomes, and mixtures thereof.
N. The layered fluid acquisition/distribution system according to any of the preceding Paragraphs A-M above wherein the plurality of fibers of the coform fibrous structure comprises a plurality of synthetic fibers, such as regenerated cellulose fibers for example regenerated cellulose fibers selected from the group consisting of: rayon fibers, viscose fibers, lyocell fibers and mixtures thereof.
O. The layered fluid acquisition/distribution system according to any of the preceding Paragraphs A-N above wherein the plurality of fibers of the coform fibrous structure comprises a plurality of natural cellulose fibers, such as natural cellulose fibers selected from the group consisting of: cotton fibers, cotton linters, trichomes, seed hairs, rice straw fibers, wheat straw fibers, bamboo fibers, bagasse fibers and mixtures thereof.
P. The layered fluid acquisition/distribution system according to any of the preceding Paragraphs A-O above wherein at least a portion of the plurality of fibers at least partially penetrate into the base nonwoven substrate.
Q. The layered fluid acquisition/distribution system according to any of the preceding Paragraphs A-P above wherein the base nonwoven substrate comprises one or more bond sites that bond the base nonwoven substrate to the coform fibrous structure, for example wherein at least one of the one or more bond sites comprise a thermal bond and/or an adhesive bond and/or a mechanical bond.
R. The layered fluid acquisition/distribution system according to any of the preceding Paragraphs A-Q above wherein the coform fibrous structure further comprises a plurality of superabsorbent polymer particles, such as superabsorbent polymer particles are derived from acrylic acid and/or superabsorbent polymer particles comprising recycled material and/or superabsorbent polymer particles comprising compostable materials and/or superabsorbent polymer particles comprising biodegradable materials and/or superabsorbent polymer particles comprising water-insoluble superabsorbent polymer particles and/or superabsorbent polymer particles comprising water-soluble superabsorbent polymer particles and/or superabsorbent polymer particles comprises water-swellable superabsorbent polymer particles and/or superabsorbent polymer particles comprising at least one superabsorbent polymer particle that exhibits a particle size that is at least two times greater than the particle size of at least one other superabsorbent polymer particle within the plurality of superabsorbent polymer particles, for example superabsorbent polymer particles comprising at least one superabsorbent polymer particle that exhibits a particle size that is at least three times greater than the particle size of at least one other superabsorbent polymer particle within the plurality of superabsorbent polymer particles.
S. The layered fluid acquisition/distribution system according to any of the preceding Paragraphs A-R above wherein the coform fibrous structure further comprises a plurality of superabsorbent polymer fibers, such as superabsorbent polymer fibers derived from acrylic acid and/or superabsorbent polymer fibers comprising recycled material and/or superabsorbent polymer fibers comprising compostable materials and/or superabsorbent polymer fibers comprising biodegradable materials and/or superabsorbent polymer fibers comprising water-insoluble superabsorbent polymer fibers and/or superabsorbent polymer fibers comprising water-soluble superabsorbent polymer fibers and/or superabsorbent polymer particles comprising water-swellable superabsorbent polymer fibers and/or superabsorbent polymer particles comprising at least one superabsorbent polymer fiber that exhibits an average diameter that is at least two times greater than the average diameter of at least one other superabsorbent polymer fiber within the plurality of absorbent polymer fibers, for example superabsorbent polymer fibers comprising at least one superabsorbent polymer fiber that exhibits an average diameter that is at least three times greater than the average diameter of at least one other superabsorbent polymer fiber within the plurality of superabsorbent polymer fibers.
T. The layered fluid acquisition/distribution system according to any of the preceding Paragraphs A-S above wherein the coform fibrous structure further comprises a plurality of superabsorbent polymer particles that are commingled with the plurality of filaments and the plurality of fibers.
U. The layered fluid acquisition/distribution system according to any of the preceding Paragraphs A-T above wherein the coform fibrous structure further comprises a plurality of superabsorbent polymer fibers that are commingled with the plurality of filaments and the plurality of fibers.
V. The layered fluid acquisition/distribution system according to any of the preceding Paragraphs A-U above wherein the coform fibrous structure comprises a plurality of water-insoluble particles.
W. The layered fluid acquisition/distribution system according to any of the preceding Paragraphs A-V above wherein the coform fibrous structure comprises a plurality of water-soluble particles.
X. The layered fluid acquisition/distribution system according to any of the preceding Paragraphs A-W above wherein the coform fibrous structure is a pre-formed coform fibrous structure.
Y. The layered fluid acquisition/distribution system according to any of the preceding Paragraphs A-X above wherein the coform fibrous structure is direct formed onto the base nonwoven structure wherein the plurality of filaments are spun from a die and directly laid on a surface of the base nonwoven substrate.
Z. An absorbent article comprising:
   a. a topsheet;
   b. a layered fluid acquisition/distribution system according to any of the preceding Paragraphs A-Y above.
AA. The absorbent article according to Paragraph Z above wherein the absorbent article further comprises a backsheet.
BB. The absorbent article according to Paragraph AA above wherein the layered fluid acquisition/distribution system is positioned between the topsheet and the backsheet.
CC. The absorbent article according to any of the preceding Paragraphs Z-BB above wherein the layered fluid acquisition/distribution system further comprises a fluid storage system.
DD. The absorbent article according to Paragraph CC above wherein the layered fluid acquisition/distribution system is positioned between the topsheet and the fluid storage system.
EE. The absorbent article according to any of the preceding Paragraphs CC-DD above wherein the topsheet, the layered fluid acquisition/distribution system, and fluid storage system are arranged within the absorbent article such that a capillarity cascade is created such that fluid moves from the topsheet to the layered acquisition/distribution system to the fluid storage system.
FF. The absorbent article according to any of the preceding Paragraphs Z-EE above wherein the layered fluid acquisition/distribution system comprise an absorbent core.
GG. The absorbent article according to any of the preceding Paragraphs Z-FF above wherein a fluid storage system is associated with the layered fluid acquisition/distribution system in the form of an absorbent core structure.

### Test Methods

Unless otherwise specified, all tests described herein including those described under the Definitions section and the following test methods are conducted on samples that have been conditioned in a conditioned room at a temperature of 23° C.±1.0° C. and a relative humidity of 50%±2% for a minimum of 24 hours prior to the test. These will be considered standard conditioning temperature and humidity. All plastic and paper board packaging absorbent articles, if any, must be carefully removed from the samples prior to testing. The samples tested are “usable units.” “Usable units” as used herein means absorbent articles, sheets, flats from roll stock, pre-converted flats, fibrous structures, layered fluid acquisition/distribution systems and/or coform fibrous structures. Except where noted all tests are conducted in such conditioned room, under the same environmental conditions in such conditioned room. Discard any damaged samples. Do not test samples that have defects such as wrinkles, tears, holes, and like. All instruments are calibrated according to manufacturer's specifications. The stated number of replicate samples to be tested is the minimum number.

### Basis Weight Test Method

Basis weight of an absorbent article and/or layered fluid acquisition/distribution system and/or base nonwoven substrate and/or coform fibrous structure and/or fibrous structure and/or scrim is measured on stacks of eight to twelve usable units using a top loading analytical balance with a resolution of ±0.001 g. A precision cutting die, measuring 8.890 cm by 8.890 cm or 10.16 cm by 10.16 cm is used to prepare all samples.

Condition samples under the standard conditioning temperature and humidity for a minimum of 10 minutes prior to cutting the sample. With a precision cutting die, cut the samples into squares. Combine the cut squares to form a stack eight to twelve samples thick. Measure the mass of the sample stack and record the result to the nearest 0.001 g.

$$Basis Weight , g / {m}^{2} = \frac{mass of stack}{( area of 1 square in stack ) ( \# squares in stack )} \qquad Calculation$$

Report result to the nearest 0.1 g/m². Sample dimensions can be changed or varied using a similar precision cutter as mentioned above, so as at least 645 square centimeters of sample area is in the stack.

Individual layered fluid acquisition/distribution systems and/or base nonwoven substrates and/or coform fibrous structures and/or fibrous structures and/or scrims (collectively referred to as “components” for purposes of this test) that are ultimately combined to form an absorbent article may be collected during their respective making operation prior to combining with other components and then the basis weight of the respective component is measured as outlined above.

### Average Diameter Test Method

There are many ways to measure the average diameter of a fibrous element and/or plurality of fibrous elements and/or one or more fiber and/or one or more filaments. One way is by optical measurement. An article and/or fibrous structure comprising fibrous elements is cut into a rectangular shape sample, approximately 20 mm by 35 mm. The sample is then coated using a SEM sputter coater (EMS Inc, PA, USA) with gold so as to make the fibrous elements relatively opaque. Typical coating thickness is between 50 and 250 nm. The sample is then mounted between two standard microscope slides and compressed together using small binder clips. The sample is imaged using a 10× objective on an Olympus BHS microscope with the microscope light-collimating lens moved as far from the objective lens as possible. Images are captured using a Nikon D1 digital camera. A Glass microscope micrometer is used to calibrate the spatial distances of the images. The approximate resolution of the images is 1 μm/pixel. Images will typically show a distinct bimodal distribution in the intensity histogram corresponding to the filaments and the background. Camera adjustments or different basis weights are used to achieve an acceptable bimodal distribution. Typically 10 images per sample are taken and the image analysis results averaged.

The images are analyzed in a similar manner to that described by B. Pourdeyhimi, R. and R. Dent in “Measuring fiber diameter distribution in nonwovens” (Textile Res. J. 69 (4) 233-236, 1999). Digital images are analyzed by computer using the MATLAB (Version. 6.1) and the MATLAB Image Processing Tool Box (Version 3.) The image is first converted into a grayscale. The image is then binarized into black and white pixels using a threshold value that minimizes the intraclass variance of the thresholded black and white pixels. Once the image has been binarized, the image is skeletonized to locate the center of each fibrous element in the image. The distance transform of the binarized image is also computed. The scalar product of the skeletonized image and the distance map provides an image whose pixel intensity is either zero or the radius of the fibrous element at that location. Pixels within one radius of the junction between two overlapping fibrous elements are not counted if the distance they represent is smaller than the radius of the junction. The remaining pixels are then used to compute a length-weighted histogram of fibrous element diameters contained in the image.

### Caliper Test Method

Caliper at 0.69 KPa can be performed on an absorbent article and/or a layered fluid acquisition/distribution system and/or a base nonwoven substrate and/or a coform fibrous structure and/or a fibrous structure and/or a scrim. If measured on something other than the absorbent article, the component and/or components of interest from the absorbent article are measured before assembling into the absorbent article and/or are disassembled from the absorbent article (separated from one or more other components) using cryo-spray as needed. Samples are conditioned at 23° C.±3 C° and 50%±2% relative humidity for two hours prior to testing. Unless otherwise specified calipers are performed centered at the visibly identifiable zones.

The caliper of an absorbent article test sample is measured using a calibrated digital linear caliper (e.g., Ono Sokki GS-503 or equivalent fitted with a 24.2 mm diameter foot with an anvil that is large enough that the absorbent article test sample can lie flat. The foot applies a confining pressure of 0.69 KPa to the absorbent article test sample. Zero the caliper foot against the anvil. Lift the foot and insert the absorbent article test sample flat against the anvil with the body facing side facing upward and the site of interest centered under the foot. Lower the foot at about 5 mm/sec onto the absorbent article test sample. Read the caliper (mm) 5.0 sec after resting the foot on the absorbent article test sample and record to the nearest 0.01 mm.

### Air Permeability Test Method

The air permeability of an absorbent article and/or a layered fluid acquisition/distribution system and/or a base nonwoven substrate and/or a coform fibrous structure and/or a fibrous structure and/or a scrim is measured using the European Disposables and Nonwovens Association (EDANA) 140.2-99 Test Method with the following modifications: 1) analysis area is 38.3 cm², 2) pressure drop is 125 Pa, and 3) report in m³/m²/min units to the nearest 1 m³/m²/min.

### Wet and Dry CD and MD 3 Point Bend Test Method

The bending properties of an absorbent article test sample are measured on a universal constant rate of extension test frame (a suitable instrument is the MTS Alliance using TestSuite Software, as available from MTS Systems Corp., Eden Prairie, MN, or equivalent) equipped with a load cell for which the forces measured are within 1% to 99% of the limit of the cell. The test is executed on dry absorbent article test samples as well as wet absorbent article test samples. The intention of this method is to mimic deformation created in the x-y plane by a wearer of an absorbent article during normal use. All testing is performed in a room controlled at 23° C.±3° C. and 50%±2% relative humidity.

The bottom stationary fixture consists of two cylindrical bars 3.175 mm in diameter by 110 mm in length, made of polished stainless steel each mounted on each end with frictionless roller bearings. These 2 bars are mounted horizontally, aligned front to back and parallel to each other, with top radii of the bars vertically aligned and are free to rotate around the diameter of the cylinder by the frictionless bearings. Furthermore, the fixture allows for the two bars to be move horizontally away from each other on a track so that a gap can be set between them while maintaining their orientation. The top fixture consists of a third cylinder bar also 3.175 mm in diameter by 110 mm in length, made of polished stainless steel mounted on each end with frictionless roller bearings. When in place the bar of the top fixture is parallel to and aligned front to back with the bars of the bottom fixture and is centered between the bars if the bottom fixture. Both fixtures include an integral adapter appropriate to fit the respective position on the universal test frame and lock into position such that the bars are orthogonal to the motion of the crossbeam of the test frame.

Set the gap (“Span”) between the bars of the lower fixture to 25 mm±0.5 mm (center of bar to center of bar) with the upper bar centered at the midpoint between the lower bars. Set the gage (bottom of top bar to top of lower bars) to 1.0 cm.

The thickness (“caliper”) of the absorbent article test sample is measured using a manually-operated micrometer equipped with a pressure foot capable of exerting a steady pressure of 0.1 psi±0.01 psi. The manually-operated micrometer is a dead-weight type instrument with readings accurate to 0.01 mm. A suitable instrument is Mitutoyo Series 543 ID-C Digimatic, available from VWR International, or equivalent. The pressure foot is a flat circular moveable face with a diameter no greater than 25.4 mm. The absorbent article test sample is supported by a horizontal flat reference platform that is larger than and parallel to the surface of the pressure foot. Zero the micrometer against the horizontal flat reference platform. Place the absorbent article test sample onto the platform, centered beneath the pressure foot. The pressure foot is lowered by hand with a descent rate of 3±1 mm/s until the full weight of the pressure is exerted onto the absorbent article test sample. After 5 seconds elapse, the thickness is recorded as caliper to the nearest 0.01 mm.

The test fluid used to dose the wet absorbent article test sample is prepared by adding 100.0 grams of sodium chloride (reagent grade, any convenient source) to 900 grams of deionized water in a 1-liter Erlenmeyer flask. Agitate until the sodium chloride is completely dissolved.

The absorbent article samples are conditioned at 23° C.±3° C. and 50%±2% relative humidity two hours prior to testing. Dry absorbent article test samples are taken from an area of the sample that is free from any seams and residua of folds or wrinkles, and ideally from the center of absorbent article (intersection of longitudinal and lateral midlines). The dry absorbent article test samples are prepared for MD (machine direction) bending by cutting them to a width of 50.8 mm along the CD (cross direction; parallel to the lateral axis of the sample) and a length of 50.8 mm along the MD (parallel to the longitudinal axis of the sample), maintaining their orientation after they are cut, and marking the body-facing surface (or the surface intended to face the body of a finished article). The dry absorbent article samples are prepared for CD (machine direction) bending by cutting them to a width of 50.8 mm along the MD (cross direction; parallel to the lateral axis of the sample) and a length of 50.8 mm along the CD (parallel to the longitudinal axis of the sample), maintaining their orientation after they are cut, and marking the body-facing surface (or the surface intended to face the body of a finished article). Measure the thickness of the absorbent article test sample, as described herein, and record as dry absorbent article test sample caliper to the nearest 0.01 mm. Now measure the mass of the absorbent article test sample and record as dry mass to the nearest 0.001 grams. Calculate the basis weight of the absorbent article test sample by dividing the mass (g) by the area (0.002581 m²) and record as dry absorbent article test sample basis weight to the nearest 0.01 g/m². Calculate the bulk density of the absorbent article test sample by dividing the absorbent article test sample basis weight (g/m²) by the absorbent article test sample thickness (mm), then dividing the quotient by 1000, and record as dry absorbent article test sample density to the nearest 0.01 g/cm³. In like fashion, five replicate dry absorbent article test samples are prepared.

Wet absorbent article test samples are initially prepared in the exact manner as for the dry absorbent article test sample, followed by the addition of test fluid just prior to testing, as follows. First, the thickness and mass of the dry absorbent article test sample is measured, as described herein, and recorded as initial thickness to the nearest 0.01 mm and initial mass to the nearest 0.001 g. Next, the dry absorbent article test sample is fully submersed in the test fluid for 60 seconds. After 60 seconds elapse, the absorbent article test sample is removed from the test fluid and oriented vertically for 30 seconds to allow any excess fluid to drip off. Now the thickness and mass of the wet absorbent article test sample are measured, as described herein, and recorded as wet absorbent article test sample caliper to the nearest 0.01 mm and wet absorbent article test sample mass to the nearest 0.001 g. If desired, the mass of test fluid in the absorbent article test sample is calculated by subtracting the initial mass (g) from the wet absorbent article test sample mass (g) and recording as absorbent article test sample fluid amount to the nearest 0.001 g. After the wet absorbent article test sample is removed from the test fluid, it must be tested within 10 minutes. In like fashion, five replicate wet absorbent article test samples are prepared.

Program the universal test frame for a flexural bend test, to move the crosshead such that the top fixture moves down with respect to the lower fixture at a rate of 1.0 mm/sec until the upper bar touches the top surface of the absorbent article test sample with a nominal force of 0.02 N, then continue for an additional 12 mm. The crosshead is then immediately returned to the original gage at a rate of 1.0 mm/s. Force (N) and displacement (mm) data are continuously collected at 100 Hz throughout the test.

Load a dry absorbent article test sample such that it spans the two lower bars and is centered under the upper bar, with its sides parallel to the bars. For MD bending, the MD direction of the absorbent article test sample is perpendicular to the length of the 3 bars. Start the test and continuously collect force and displacement data.

Construct a graph of force (N) versus displacement (mm). From the graph, determine the maximum peak force and record as dry MD peak load to the nearest 0.01 N. Now calculate the maximum slope of the curve between initial force and maximum force (during the loading portion of the curve) and record to the nearest 0.1 unit. Calculate the modulus as follows, and record as dry MD modulus to the nearest 0.001 N/mm².

CD or MD Dry or Wet Bending Modulus (N/mm²)=(Slope×(Span³))/(4×absorbent article test sample width×(absorbent article test sample caliper³))

Calculate bending stiffness as follows, and record as dry MD bending stiffness to the nearest 0.1 N·mm².

CD or MD Dry or Wet Bending Stiffness (N·mm²)=Modulus×Moment of Inertia where Moment of Inertia (mm⁴)=(absorbent article test sample width×(absorbent article test sample caliper³))/12

In like fashion, the procedure is repeated for all five replicates of the dry absorbent article test samples. The arithmetic mean among the five replicate dry absorbent article test samples is calculated for each of the parameters and reported as Dry absorbent article test sample ‘Caliper’ to the nearest 0.01 mm, Dry absorbent article test sample Basis Weight to the nearest 0.01 g/m², Dry absorbent article test sample Density to the nearest 0.001 g/cm³, Dry CD or MD Peak Load to the nearest 0.01 N, Dry CD or MD Bending Modulus to the nearest 0.001 N/mm², and Dry CD or MD Bending Stiffness to the nearest N·mm².

The overall procedure is now repeated for all five replicates of the wet absorbent article test samples, reporting results as Wet CD or MD Peak Load to the nearest 0.01 N, Wet CD or MD Bending Modulus to the nearest 0.001 N/mm², and Wet CD or MD Bending Stiffness to the nearest N·mm².

### Wet and Dry Bunched Compression Test Method

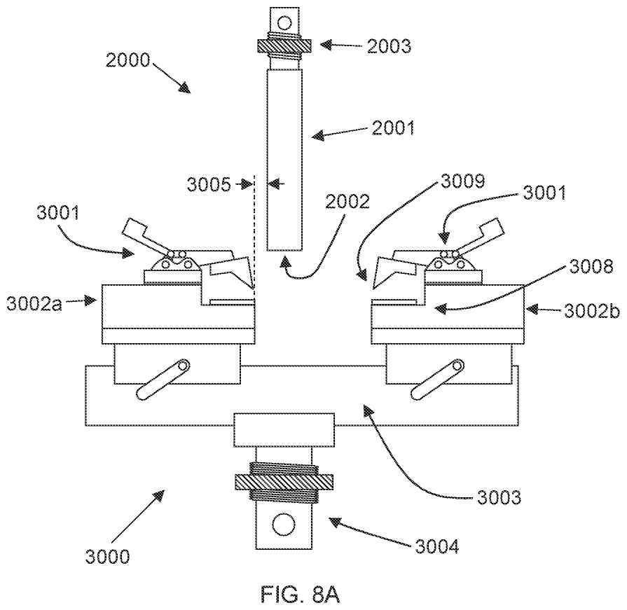

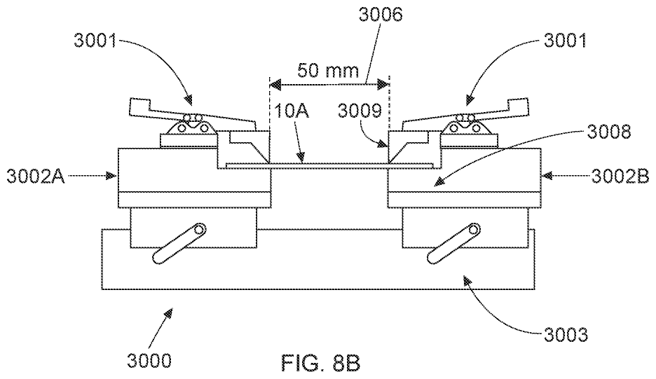

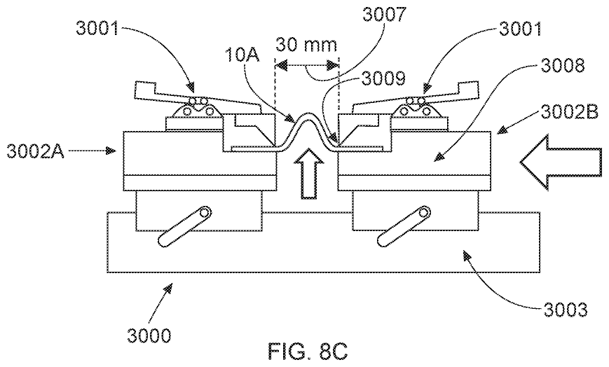

As shown in FIGS. 8A-8C, The wet and dry bunched compression test method measures the force versus displacement behavior across five cycles of load application (“compression”) and load removal (“recovery”) of an absorbent article test sample 10A that has been intentionally “bunched”, using a universal constant rate of extension test frame (a suitable instrument is the MTS Alliance using TestSuite software, as available from MTS Systems Corp., Eden Prairie, MN, or equivalent) equipped with a load cell for which the forces measured are within 1% to 99% of the limit of the cell (collectively, the “test apparatus”). The test is executed on dry absorbent article test samples 10A as well as wet absorbent article test samples 10A that are dosed with a specific amount of a test fluid. The intention of this test method is to mimic the deformation created in the z-plane of the crotch region of an absorbent article 10, or components thereof, as it is worn by the wearer during sit-stand movements. All testing is performed in a room controlled at 23□C □□3 C□□ and 50% □□2% relative humidity.

The test apparatus is depicted in FIGS. 8A-8C. The bottom stationary fixture 3000 consists of two matching sample clamps 3001 each 100 mm wide, each mounted on its own movable platform 3002A, 3002B. Each sample clamp 3001 has a “knife edge” 3009 that is 110 mm long, which clamps against a 1 mm thick hard rubber face 3008. When closed, the sample clamps 3001 are flush with the interior side of their respective platforms 3002A, 3002B. The sample clamps 3001 are aligned such that they hold an un-bunched absorbent article test sample 10A horizontal and orthogonal to the pull axis of the test apparatus. The platforms 3002A, 3002B are mounted on a rail 3003 which allows them to be moved horizontally left to right and locked into position. The rail 3003 has an adapter 3004 compatible with the mount of the test apparatus capable of securing the rail 3003 and platforms 3002A, 3002B horizontally and orthogonal to the pull axis of the test apparatus. The upper fixture 2000 is a cylindrical plunger 2001 having an overall length of 70 mm with a diameter of 25.0 mm. The contact surface 2002 is flat with no curvature. The plunger 2001 has an adapter 2003 compatible with the mount on the load cell capable of securing the plunger 2001 orthogonal to the pull axis of the test apparatus.

Prepare the absorbent article test samples 10A as follows. When testing an intact absorbent article 10, remove any release paper from any panty fastening adhesive on the garment facing side of the absorbent article 10, if present. Lightly apply talc powder to the adhesive to mitigate any tackiness. If there are cuffs, excise them with scissors so as not to disturb the topsheet or any other underlying layers of the absorbent article 10. Place the absorbent article 10, body facing surface up, on a benchtop. On the absorbent article 10, mark the intersection of the longitudinal midline and the lateral midline. Using a rectangular cutting die or equivalent cutting means, cut an absorbent article 10 test sample 100 mm in the longitudinal direction by 80 mm in the lateral direction, centered at the intersection of the midlines. When testing a material layer or layered components from an absorbent article 10, place the material layer or layered components on a benchtop and orient as it would be integrated into a finished absorbent article 10, i.e., identify the body facing surface and the lateral and longitudinal axis. Using a rectangular cutting die, or equivalent cutting means, cut an absorbent article test sample 10A from an absorbent article 10 100 mm in the longitudinal direction by 80 mm in the lateral direction, centered at the intersection of the midlines. The absorbent article test samples 10A are conditioned at 23□C □□3 C□□ and 50% □□2% relative humidity for at least 2 hours before testing. Measure the mass of the absorbent article test sample 10A and record to the nearest 0.001 grams. Calculate the basis weight of the absorbent article test sample 10A by dividing the mass (g) by the area (0.008 m²) and record as basis weight to the nearest 1 g/m².

The absorbent article test samples 10A can be analyzed both wet and dry. The dry absorbent article test sample 10A requires no further preparation. The test fluid used to dose the wet absorbent article test sample 10A is prepared by adding 100.0 grams of sodium chloride (reagent grade, any convenient source) to 900 grams of deionized water in a 1-liter Erlenmeyer flask. Agitate until the sodium chloride is completely dissolved. The wet absorbent article test sample 10A is dosed with a total of 7 ml of the test fluid as detailed below

The test fluid dose is added using a calibrated Eppendorf-type pipettor, spreading the test fluid over the complete body facing surface of the absorbent article test sample 10A within a period of approximately 3 sec. The wet absorbent article test sample 10A is then tested 10.0 min±0.1 min after the test fluid dose is applied.

Program the test apparatus to zero the load cell, then lower the upper fixture at 2.00 mm/sec until the contact surface of the plunger touches the absorbent article test sample 10A and 0.02 N is read at the load cell. Zero the crosshead. Program the system to lower the crosshead 15.00 mm at 2.00 mm/sec then immediately raise the crosshead 15.00 mm at 2.00 mm/sec. This cycle is repeated for a total of five cycles, with no delay between cycles. Data is collected at 50 Hz during all compression/decompression cycles.

As shown in FIG. 8A, position the left platform 3002A 2.5 mm from the side of the plunger 2001 (distance 3005). Lock the left platform 3002A into place. The left platform 3002A will remain stationary throughout the test. Align the right platform 3002B 50.0 mm from the closest edge of the clamp 3001 of the stationary left platform 3002A (distance 3006) as shown in FIG. 8B. Raise the plunger 2001 such that it will not interfere with loading the absorbent article test sample 10A. Open both clamps 3001. Referring to FIG. 8B, place a dry absorbent article test sample 10A with its longitudinal edges (i.e., the 100 mm long edges) within the clamps 3001 of both platforms 3002A, 3002B. With the dry absorbent article test sample 10A laterally centered, securely fasten both longitudinal edges in the clamps 3001. Referring to FIG. 8C, move the right platform 3002B toward the stationary left platform 3002A a distance of 20.0 mm so that a separation of 30.0 mm (distance 3007) between the left and right clamps 3001 of their respective left and right platforms 3002A, 3002B is achieved. Allow the dry absorbent article test sample 10A to bow upward as the movable right platform 3002B is moved into position. Now manually lower the plunger 2001 (as shown in FIG. 8A) until the contact surface 2002 (as shown in FIG. 8A) is approximately 1 cm above the top 3010 of the bowed absorbent article test sample 10A.

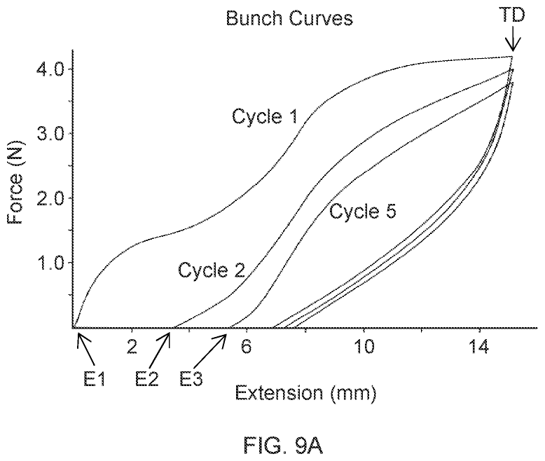

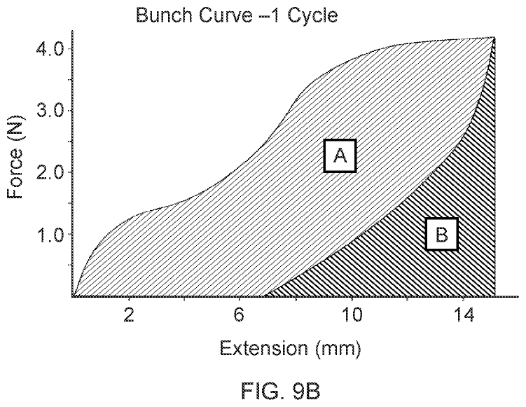

Start the test and continuously collect force (N) versus displacement or extension (mm) data for all five cycles. Construct a graph of force (N) versus displacement or extension (mm) separately for all cycles. A representative curve is shown in FIG. 9A. From the curve, determine the Dry Maximum Compression Force for each Cycle to the nearest 0.01 N, then multiply by 101.97 and record to the nearest 1 gram-force. Calculate the Dry % Recovery between the First and Second cycle as (TD−E2)/(TD−E1)*100 where TD is the total displacement or extension (mm) and E2 is the displacement or extension (mm) on the second compression curve that exceeds 0.02 N, and record to the nearest 0.01%. In like fashion calculate the Dry % Recovery between the First Cycle and other cycles as (TD−E1)/(TD−E1)*100 and record to the nearest 0.01%. Referring to FIG. 9B, calculate the Dry Energy of Compression for Cycle 1 as the area under the compression curve (i.e., area A+B) and record to the nearest 0.1 N*mm. Calculate the Dry Energy Loss from Cycle 1 as the area between the compression and decompression curves (i.e., Area A) and record to the nearest 0.1 N*mm. Calculate the Dry Energy of Recovery for Cycle 1 as the area under the decompression curve (i.e., Area B) and report to the nearest 0.1 N*mm. In like fashion calculate the Dry Energy of Compression (N*mm), Dry Energy Loss (N*mm) and Dry Energy of Recovery (N*mm) for each of the other cycles and record to the nearest 0.1 N*mm. In like fashion, analyze a total of five replicate dry absorbent article test samples 10A and report the arithmetic mean among the five dry replicates for each parameter as previously described, including basis weight.

The overall procedure is now repeated for a total of five replicate wet absorbent article test samples 10A, reporting results for each of the five cycles as the arithmetic mean among the five wet replicates for Wet Maximum Compression Force to the nearest 1 gram-force for each cycle, Wet Energy of Compression to the nearest 0.1 N*mm for each cycle, Wet Energy Loss to the nearest 0.1 N*mm for each cycle, Wet Energy of Recovery to the nearest 0.1 N*mm for each cycle and Wet % recovery for each cycle. Of particular importance is the 5th cycle wet energy of recovery and 5th cycle wet % recovery properties from this test method.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A layered fluid acquisition/distribution system comprising:
a. a base nonwoven substrate layer that comprises at least one of a carded base nonwoven substrate, spunlace base nonwoven substrate, hydroentangled base nonwoven substrate, or a spunbond base nonwoven substrate; and
b. a coform fibrous structure layer comprising a plurality of filaments and a plurality of fibers;
wherein the plurality of filaments and the plurality of fibers are comingled together.

2. The layered fluid acquisition/distribution system according to claim 1 wherein the base nonwoven substrate layer exhibits a basis weight of from about 10 gsm to about 60 gsm.

3. The layered fluid acquisition/distribution system according to claim 1 wherein the base nonwoven substrate layer comprises a plurality of fibrous elements.

4. The layered fluid acquisition/distribution system according to claim 3 wherein the plurality of fibrous elements of the base nonwoven substrate layer comprise synthetic fibers.

5. The layered fluid acquisition/distribution system according to claim 4 wherein the synthetic fibers comprise regenerated cellulose fibers.

6. The layered fluid acquisition/distribution system according to claim 5 wherein the regenerated cellulose fibers are selected from the group consisting of: rayon fibers, viscose fibers, lyocell fibers and mixtures thereof.

7. The layered fluid acquisition/distribution system according to claim 3 wherein the plurality of fibrous elements of the base nonwoven substrate layer exhibit an average diameter of from about 10 μm to about 50 μm as measured according to the Average Diameter Test Method.

8. The layered fluid acquisition/distribution system according to claim 1 wherein the base nonwoven substrate layer exhibits a caliper of from about 0.2 mm to about 1 mm as measured according to the Caliper Test Method.

9. The layered fluid acquisition/distribution system according to claim 1 wherein the base nonwoven substrate layer exhibits an air permeability of between about 150 m³/m²/min and about 500 m³/m²/min as measured according to the Air Permeability Test Method.

10. The layered fluid acquisition/distribution system according to claim 1 wherein the base nonwoven substrate layer comprises a carded base nonwoven substrate.

11. The layered fluid acquisition/distribution system according to claim 1 wherein the base nonwoven substrate layer comprises a spunlace base nonwoven substrate.

12. The layered fluid acquisition/distribution system according to claim 1 wherein the base nonwoven substrate layer comprises a hydroentangled base nonwoven substrate.

13. The layered fluid acquisition/distribution system according to claim 1 wherein the base nonwoven substrate layer comprises a spunbond base nonwoven substrate.

14. The layered fluid acquisition/distribution system according to claim 1 wherein the plurality of fibers of the coform fibrous structure comprises a plurality of pulp fibers.

15. The layered fluid acquisition/distribution system according to claim 1 wherein the plurality of fibers of the coform fibrous structure comprises a plurality of synthetic fibers.

16. The layered fluid acquisition/distribution system according to claim 15 wherein the plurality of synthetic fibers of the coform fibrous structure comprises a plurality of regenerated cellulose fibers.

17. The layered fluid acquisition/distribution system according to claim 16 wherein the regenerated cellulose fibers are selected from the group consisting of: rayon fibers, viscose fibers, lyocell fibers and mixtures thereof.

18. The layered fluid acquisition/distribution system according to claim 1 wherein at least a portion of the plurality of fibers at least partially penetrate into the base nonwoven substrate.

19. An absorbent article comprising:
a. a topsheet;
b. a layered fluid acquisition/distribution system according to claim 1.

20. The absorbent article according to claim 19 wherein the absorbent article further comprises a backsheet.

21. The absorbent article according to claim 20 wherein the layered fluid acquisition/distribution system is positioned between the topsheet and the backsheet.

22. The absorbent article according to claim 19 wherein the layered fluid acquisition/distribution system further comprises a fluid storage system.

23. The absorbent article according to claim 22 wherein the layered fluid acquisition/distribution system is positioned between the topsheet and the fluid storage system.

24. The absorbent article according to claim 22 wherein the topsheet, the layered fluid acquisition/distribution system, and fluid storage system are arranged within the absorbent article such that a capillarity cascade is created such that fluid moves from the topsheet to the layered acquisition/distribution system to the fluid storage system.

25. A process for making a layered fluid acquisition/distribution system comprising the steps of:
a) providing a base nonwoven substrate having a surface, wherein the base nonwoven substrate comprises at least one of a carded base nonwoven substrate, spunlace base nonwoven substrate, hydroentangled base nonwoven substrate, or a spunbond base nonwoven substrate;
b) spinning a plurality of filaments from a die;
c) forming a coform mixture by mixing a plurality of fibers with the plurality of filaments, wherein the filaments and fibers are commingled together forming a mixture of filaments and fibers;
d) collecting the coform mixture on a surface of the base nonwoven substrate such that a coform fibrous structure is formed on the surface of the base nonwoven substrate.
